(19) United States
(12) Patent Application Publication
(10) Pub. No.: US 2013/0123261 A1
(43) Pub. Date: May 16, 2013

(54) ISOXAZOLINE COMPOUNDS IN TYPE 2 DIABETES AND OTHER MALADIES

(71) Applicants: Abhay Satoskar, Upper Arlington, OH (US); Thais Sieleck-Dzurdz, Kennett Square, PA (US)

(72) Inventors: Abhay Satoskar, Upper Arlington, OH (US); Thais Sieleck-Dzurdz, Kennett Square, PA (US)

(73) Assignee: CPSI STOCKHOLDER TRUST, New York, NY (US)

(21) Appl. No.: 13/512,297

(22) PCT Filed: Nov. 26, 2010
(86) PCT No.: PCT/US10/58137

§ 371 Date: Jan. 28, 2013

(30) Foreign Application Priority Data
Nov. 25, 2009 (US) 61264620

(51) Int. Cl. A61K 31/42; A61K 31/5377; A61K 31/496

(52) U.S. Cl. CPC A61K 31/42; A61K 31/496; A61K 31/5377
USPC 514/236.8; 514/254.04; 514/378

(57) ABSTRACT

Methods for treating diabetes in a subject, reducing the blood glucose level of a subject suffering from diabetes, and reducing or preventing an increase in the level of resistin in a subject, comprising administering to said subject a compound having the following formula: wherein R¹, R², R³, R⁴, R⁵, X, Y, and R¹⁶ are described herein; or salt thereof, prodrug thereof, or combination thereof, optionally in contact with one or more pharmaceutical carrier.

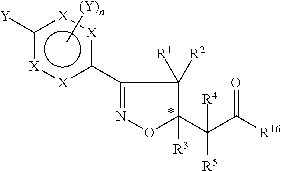

### FIELD OF THE APPLICATION

The present application relates to the use of isoxazoline compounds in Type 2 diabetes (non-insulin dependent diabetes mellitus) and other maladies.

### BACKGROUND

Non-insulin dependent diabetes mellitus (NIDDM), also known as type 2 diabetes mellitus, is a global health problem. Although NIDDM is more likely to occur in obese individuals, genetic background and environmental factors also influence the development of this disease. Several studies have shown that development of NIDDM is associated with impaired responsiveness to insulin and subsequent failure of pancreatic β cells to secrete adequate amounts of insulin required to maintain blood glucose level.

Macrophage migration inhibitory factor (MIF) was among one of the first cytokines to be described in the late 1960's. MIF is a pleiotropic molecule that is ubiquitously produced during inflammatory responses by many cells, including activated T cells, macrophages and the pituitary gland. MIF promotes the production of inflammatory Th1 cytokines including TNF, IFN-γ, IL-2 and IL-6 and inhibits anti-inflammatory effects of corticosteroids. Levels of MIF are elevated in patients suffering from inflammatory autoimmune diseases, such as arthritis (1-3) and chronic colitis (4). Furthermore, several experimental studies using MIF−/− mice and anti-MIF blocking antibodies have shown that MIF is involved in pathogenesis of inflammatory diseases such as collagen type II-induced arthritis (5), immunologically induced kidney diseases (6) and colitis (4). Several clinical studies have shown that serum levels of MIF are elevated in patients suffering from insulin-dependent diabetes mellitus (IDDM; also known as type I diabetes) and NIDDM (7-9). Recent experimental studies using anti-MIF Abs as well MIF−/− mice have shown that MIF is necessary for progression of IDDM (10;11), but its role in pathogenesis of NIDDM has not been investigated.

### BRIEF DESCRIPTION OF THE FIGURES

FIG. 1 graphically shows that MIF−/− BALB/c mice develop significantly less severe NIDDM as compared to WT BALB/c mice following STZ injection.

FIG. 2 graphically shows that MIF−/− BALB/c mice display better glucose tolerance than WT mice.

FIG. 3 graphically shows analysis of IL-1, IL-6 and TNF-α production in WT and MIF−/− BALB/c mice following STZ-induced NIDDM.

FIG. 4 graphically shows histopathology and quantification of insulin and GLUT2 mRNA levels in pancreas of WT and MIF−/− mice.

FIG. 5 graphically shows that MIF−/− mice produce significantly less resistin as compared to WT mice.

FIG. 6 graphically shows that oral administration of MIF antagonist CPSI-1306 significantly reduces severity and progression of STZ-induced NIDDM in outbred ICR mice.

FIG. 7 graphically shows analysis of blood glucose, MIF, TNF-α and resistin levels in patients with NIDDM.

### BRIEF SUMMARY OF THE SEVERAL EMBODIMENTS

One embodiment relates to a method for treating diabetes in a subject, comprising administering to said subject a compound having the following formula:

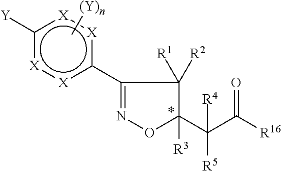

wherein at least the carbon marked “*” is chiral;

wherein R¹, R², R³, R⁴, and R⁵ are each independently hydrogen, an alkyl group, a cycloalkyl group, a halo group, an alkenyl group, an alkynyl group, a hydroxy group, an oxo group, a mercapto group, an alkylthio group, an alkoxy group, an aryl group, a heterocyclic group, a heteroaryl group, an aryloxy group, a heteroaryloxy group, an aralkyl group, a heteroaralkyl group, an aralkoxy group, a heteroaralkoxy group, an amino group, an alkylamino group, a dialkylamino group, an amidine group, an amide group, a carbamoyl group, an alkylcarbonyl group, an alkoxycarbonyl group, an alkylaminocarbonyl group, a dialkylamino carbonyl group, an arylcarbonyl group, an aryloxycarbonyl group, an alkylsulfonyl group, an arylsulfonyl group, perhaloalkyl group, a perhaloalkoxy group, a perhalocycloalkyl group, a perhaloalkenyl group, a perhaloalkynyl group, a perhaloaryl group, or a perhaloaralkyl group; wherein R¹ and R² may be taken together to form a cyclic group; wherein R⁴ and R⁵ may be taken together to form a cyclic group; wherein each group may be optionally and independently straight or branched; wherein each group may be optionally and independently substituted by one or more independent substituents; and wherein one or more than one atom in each group may be optionally and independently replaced with one or more independent heteroatoms;

wherein each X is independently carbon or nitrogen, wherein when any X is carbon, it comprises a Y substituent, n being an integer of from 1 to 4 and being the number of X's that are carbon;

wherein each Y is independently a carbonyl group, a carboxylic acid group, a carboxylate group, hydrogen, an alkyl group, a cycloalkyl group, a halo group, an alkenyl group, an alkynyl group, a hydroxy group, an oxo group, a mercapto group, an alkylthio group, an alkoxy group, an aryl group, a heterocyclic group, a heteroaryl group, an aryloxy group, a heteroaryloxy group, an aralkyl group, a heteroaralkyl group, an aralkoxy group, a heteroaralkoxy group, an amino group, an alkylamino group, a dialkylamino group, an amidine group, an amide group, a carbamoyl group, an alkylcarbonyl group, an alkoxycarbonyl group, an alkylaminocarbonyl group, a dialkylamino carbonyl group, an arylcarbonyl group, an aryloxycarbonyl group, an alkylsulfonyl group, an arylsulfonyl group, perhaloalkyl group, a perhaloalkoxy group, a perhalocycloalkyl group, a perhaloalkenyl group, a perhaloalkynyl group, a perhaloaryl group, or a perhaloaralkyl group; wherein two Y groups may be taken together to form a cyclic or aryl group; wherein each group may be optionally and independently straight or branched; wherein each group may be optionally and independently substituted by one or more independent substituents; and wherein one or more than one atom in each group may be optionally and independently replaced with one or more independent heteroatoms;

and wherein R¹⁶ is an alkyl group, a cycloalkyl group, a halo group, an alkenyl group, an alkynyl group, a hydroxy group, an oxo group, a mercapto group, an alkylthio group, an alkoxy group, an aryl group, a heterocyclic group, a heteroaryl group, an aryloxy group, a heteroaryloxy group, an aralkyl group, a heteroaralkyl group, an aralkoxy group, a heteroaralkoxy group, an amino group, an alkylamino group, a dialkylamino group, an amidine group, an amide group, a carbamoyl group, an alkylcarbonyl group, an alkoxycarbonyl group, an alkylaminocarbonyl group, a dialkylamino carbonyl group, an arylcarbonyl group, an aryloxycarbonyl group, an alkylsulfonyl group, an arylsulfonyl group, perhaloalkyl group, a perhaloalkoxy group, a perhalocycloalkyl group, a perhaloalkenyl group, a perhaloalkynyl group, a perhaloaryl group, or a perhaloaralkyl group; wherein any two alkyl groups may be taken together to form a cyclic group; wherein each group may be optionally and independently straight or branched; wherein each group may be optionally and independently substituted by one or more independent substituents; and wherein one or more than one atom in each group may be optionally and independently replaced with one or more independent heteroatoms;

or salt thereof, prodrug thereof, or combination thereof, optionally in contact with one or more pharmaceutical carrier.

One embodiment relates to a method for reducing or preventing an increase in the level of resistin in a subject, comprising administering to said subject a compound having the following formula:

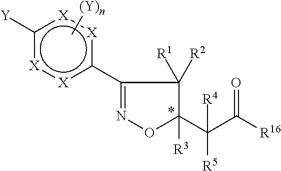

wherein at least the carbon marked “*” is chiral;

wherein R¹, R², R³, R⁴, and R⁵ are each independently hydrogen, an alkyl group, a cycloalkyl group, a halo group, an alkenyl group, an alkynyl group, a hydroxy group, an oxo group, a mercapto group, an alkylthio group, an alkoxy group, an aryl group, a heterocyclic group, a heteroaryl group, an aryloxy group, a heteroaryloxy group, an aralkyl group, a heteroaralkyl group, an aralkoxy group, a heteroaralkoxy group, an amino group, an alkylamino group, a dialkylamino group, an amidine group, an amide group, a carbamoyl group, an alkylcarbonyl group, an alkoxycarbonyl group, an alkylaminocarbonyl group, a dialkylamino carbonyl group, an arylcarbonyl group, an aryloxycarbonyl group, an alkylsulfonyl group, an arylsulfonyl group, perhaloalkyl group, a perhaloalkoxy group, a perhalocycloalkyl group, a perhaloalkenyl group, a perhaloalkynyl group, a perhaloaryl group, or a perhaloaralkyl group; wherein R¹ and R² may be taken together to form a cyclic group; wherein R⁴ and R⁵ may be taken together to form a cyclic group; wherein each group may be optionally and independently straight or branched; wherein each group may be optionally and independently substituted by one or more independent substituents; and wherein one or more than one atom in each group may be optionally and independently replaced with one or more independent heteroatoms;

wherein each X is independently carbon or nitrogen, wherein when any X is carbon, it comprises a Y substituent, n being an integer of from 1 to 4 and being the number of X's that are carbon;

wherein each Y is independently a carbonyl group, a carboxylic acid group, a carboxylate group, hydrogen, an alkyl group, a cycloalkyl group, a halo group, an alkenyl group, an alkynyl group, a hydroxy group, an oxo group, a mercapto group, an alkylthio group, an alkoxy group, an aryl group, a heterocyclic group, a heteroaryl group, an aryloxy group, a heteroaryloxy group, an aralkyl group, a heteroaralkyl group, an aralkoxy group, a heteroaralkoxy group, an amino group, an alkylamino group, a dialkylamino group, an amidine group, an amide group, a carbamoyl group, an alkylcarbonyl group, an alkoxycarbonyl group, an alkylaminocarbonyl group, a dialkylamino carbonyl group, an arylcarbonyl group, an aryloxycarbonyl group, an alkylsulfonyl group, an arylsulfonyl group, perhaloalkyl group, a perhaloalkoxy group, a perhalocycloalkyl group, a perhaloalkenyl group, a perhaloalkynyl group, a perhaloaryl group, or a perhaloaralkyl group; wherein two Y groups may be taken together to form a cyclic or aryl group; wherein each group may be optionally and independently straight or branched; wherein each group may be optionally and independently substituted by one or more independent substituents; and wherein one or more than one atom in each group may be optionally and independently replaced with one or more independent heteroatoms;

and wherein R¹⁶ is an alkyl group, a cycloalkyl group, a halo group, an alkenyl group, an alkynyl group, a hydroxy group, an oxo group, a mercapto group, an alkylthio group, an alkoxy group, an aryl group, a heterocyclic group, a heteroaryl group, an aryloxy group, a heteroaryloxy group, an aralkyl group, a heteroaralkyl group, an aralkoxy group, a heteroaralkoxy group, an amino group, an alkylamino group, a dialkylamino group, an amidine group, an amide group, a carbamoyl group, an alkylcarbonyl group, an alkoxycarbonyl group, an alkylaminocarbonyl group, a dialkylamino carbonyl group, an arylcarbonyl group, an aryloxycarbonyl group, an alkylsulfonyl group, an arylsulfonyl group, perhaloalkyl group, a perhaloalkoxy group, a perhalocycloalkyl group, a perhaloalkenyl group, a perhaloalkynyl group, a perhaloaryl group, or a perhaloaralkyl group; wherein any two alkyl groups may be taken together to form a cyclic group; wherein each group may be optionally and independently straight or branched; wherein each group may be optionally and independently substituted by one or more independent substituents; and wherein one or more than one atom in each group may be optionally and independently replaced with one or more independent heteroatoms;

or salt thereof, prodrug thereof, or combination thereof, optionally in contact with one or more pharmaceutical carrier.

One embodiment relates to a method for reducing the blood glucose level of a subject suffering from diabetes, comprising administering to said subject a compound having the following formula:

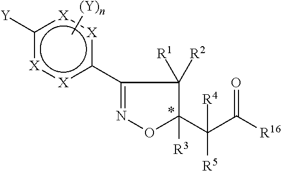

wherein at least the carbon marked “*” is chiral;

wherein R¹, R², R³, R⁴, and R⁵ are each independently hydrogen, an alkyl group, a cycloalkyl group, a halo group, an alkenyl group, an alkynyl group, a hydroxy group, an oxo group, a mercapto group, an alkylthio group, an alkoxy group, an aryl group, a heterocyclic group, a heteroaryl group, an aryloxy group, a heteroaryloxy group, an aralkyl group, a heteroaralkyl group, an aralkoxy group, a heteroaralkoxy group, an amino group, an alkylamino group, a dialkylamino group, an amidine group, an amide group, a carbamoyl group, an alkylcarbonyl group, an alkoxycarbonyl group, an alkylaminocarbonyl group, a dialkylamino carbonyl group, an arylcarbonyl group, an aryloxycarbonyl group, an alkylsulfonyl group, an arylsulfonyl group, perhaloalkyl group, a perhaloalkoxy group, a perhalocycloalkyl group, a perhaloalkenyl group, a perhaloalkynyl group, a perhaloaryl group, or a perhaloaralkyl group; wherein R¹ and R² may be taken together to form a cyclic group; wherein R⁴ and R⁵ may be taken together to form a cyclic group; wherein each group may be optionally and independently straight or branched; wherein each group may be optionally and independently substituted by one or more independent substituents; and wherein one or more than one atom in each group may be optionally and independently replaced with one or more independent heteroatoms;

wherein each X is independently carbon or nitrogen, wherein when any X is carbon, it comprises a Y substituent, n being an integer of from 1 to 4 and being the number of X's that are carbon;

wherein each Y is independently a carbonyl group, a carboxylic acid group, a carboxylate group, hydrogen, an alkyl group, a cycloalkyl group, a halo group, an alkenyl group, an alkynyl group, a hydroxy group, an oxo group, a mercapto group, an alkylthio group, an alkoxy group, an aryl group, a heterocyclic group, a heteroaryl group, an aryloxy group, a heteroaryloxy group, an aralkyl group, a heteroaralkyl group, an aralkoxy group, a heteroaralkoxy group, an amino group, an alkylamino group, a dialkylamino group, an amidine group, an amide group, a carbamoyl group, an alkylcarbonyl group, an alkoxycarbonyl group, an alkylaminocarbonyl group, a dialkylamino carbonyl group, an arylcarbonyl group, an aryloxycarbonyl group, an alkylsulfonyl group, an arylsulfonyl group, perhaloalkyl group, a perhaloalkoxy group, a perhalocycloalkyl group, a perhaloalkenyl group, a perhaloalkynyl group, a perhaloaryl group, or a perhaloaralkyl group; wherein two Y groups may be taken together to form a cyclic or aryl group; wherein each group may be optionally and independently straight or branched; wherein each group may be optionally and independently substituted by one or more independent substituents; and wherein one or more than one atom in each group may be optionally and independently replaced with one or more independent heteroatoms;

and wherein R¹⁶ is an alkyl group, a cycloalkyl group, a halo group, an alkenyl group, an alkynyl group, a hydroxy group, an oxo group, a mercapto group, an alkylthio group, an alkoxy group, an aryl group, a heterocyclic group, a heteroaryl group, an aryloxy group, a heteroaryloxy group, an aralkyl group, a heteroaralkyl group, an aralkoxy group, a heteroaralkoxy group, an amino group, an alkylamino group, a dialkylamino group, an amidine group, an amide group, a carbamoyl group, an alkylcarbonyl group, an alkoxycarbonyl group, an alkylaminocarbonyl group, a dialkylamino carbonyl group, an arylcarbonyl group, an aryloxycarbonyl group, an alkylsulfonyl group, an arylsulfonyl group, perhaloalkyl group, a perhaloalkoxy group, a perhalocycloalkyl group, a perhaloalkenyl group, a perhaloalkynyl group, a perhaloaryl group, or a perhaloaralkyl group; wherein any two alkyl groups may be taken together to form a cyclic group; wherein each group may be optionally and independently straight or branched; wherein each group may be optionally and independently substituted by one or more independent substituents; and wherein one or more than one atom in each group may be optionally and independently replaced with one or more independent heteroatoms;

or salt thereof, prodrug thereof, or combination thereof, optionally in contact with one or more pharmaceutical carrier.

For convenience, the compound having the following formula

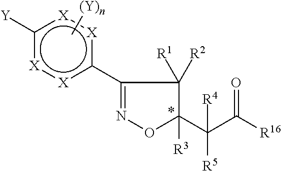

is referred to herein as the subject compound.

In one embodiment, the subject compound is selected from the following compounds:

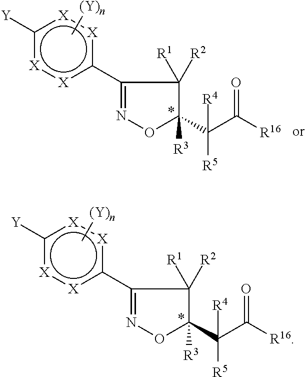

In one embodiment, the subject compound is the following compound:

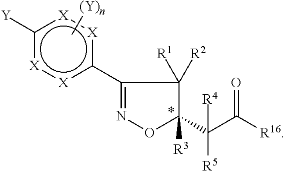

In one embodiment, the subject compound is the following compound:

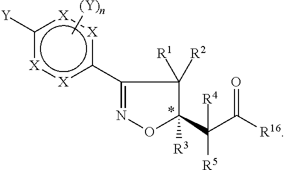

In one embodiment, R¹, R², R³, R⁴, and R⁵ are each independently hydrogen, an alkyl group, a cycloalkyl group, an alkenyl group, an alkynyl group, an oxo group, an aryl group, a heterocyclic group, a heteroaryl group, an aralkyl group, a heteroaralkyl group, an amino group, an alkylamino group, a dialkylamino group, an amidine group, an amide group, an alkylcarbonyl group, an alkoxycarbonyl group, an alkylaminocarbonyl group, a dialkylamino carbonyl group, an arylcarbonyl group, an aryloxycarbonyl group, an alkylsulfonyl group, an arylsulfonyl group, perhaloalkyl group, a perhalocycloalkyl group, a perhaloalkenyl group, a perhaloalkynyl group, a perhaloaryl group, or a perhaloaralkyl group; wherein R¹ and R² may be taken together to form a cyclic group; wherein R⁴ and R⁵ may be taken together to form a cyclic group; wherein each group may be optionally and independently straight or branched; wherein each group may be optionally and independently substituted by one or more independent substituents; and wherein one or more than one atom in each group may be optionally and independently replaced with one or more independent heteroatoms.

In one embodiment, one or both of R⁴ and R⁵ are hydrogen.

In one embodiment, only one of R⁴ and R⁵ is hydrogen.

In one embodiment, an alkyl group is a univalent, acyclic, straight or branched, substituted or unsubstituted, saturated or unsaturated, hydrocarbon radical. In one embodiment, the alkyl group has the general formula (notwithstanding optional unsaturation, substitution or the like) —CₙH₂ₙ₊₁. In one embodiment, n is 1-20 ((C₁-C₂₀) alkyl), which may suitably include C₁, C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, and C₂₀ alkyl groups. In one embodiment, the alkyl group may be straight or branched, substituted or unsubstituted, saturated or unsaturated, or any combination thereof. In one embodiment, one or more hydrogens may be optionally and independently replaced by one or more substituent groups. In one embodiment, one or more carbon atoms may be optionally and independently replaced with one or more heteroatoms such as O, S, N, B, or any combination thereof. In one embodiment, the alkyl group may contain one or more double bond, one or more triple bond, or any combination thereof. In one embodiment, the alkyl group is attached to the parent structure through one or more independent divalent intervening substituent groups. Some examples of alkyl groups, which are not intended to be limiting, include methyl, ethyl, n-propyl, isopropyl, n-butyl, iso-butyl, secondary-butyl, tertiary-butyl, and the like.

In one embodiment, a cycloalkyl group is a univalent, mono- or polycyclic, substituted or unsubstituted, saturated or unsaturated hydrocarbon radical. In one embodiment, the cycloalkyl group has the general formula (notwithstanding optional unsaturation, substitution, or the like) —CₙH₂ₙ₋₁. In one embodiment, n is 3-20 ((C₃-C₂₀) cycloalkyl), which may suitably include C₃, C₄, C₅, C₆, C₇, C₈, C₉, C₁₀, C₁₁, C₁₂, C₁₃, C₁₄, C₁₅, C₁₆, C₁₇, C₁₈, C₁₉, and C₂₀ cycloalkyl groups. In one embodiment, the cycloalkyl group is substituted or unsubstituted, saturated or unsaturated, mono-, bi-, tri-, or poly-cyclic, or any combination thereof. In one embodiment, one or more hydrogens may be optionally and independently replaced by one or more substituent groups. In one embodiment, the cycloalkyl group may have one or more sites of unsaturation, e.g., it may contain one or more double bond, one or more triple bond, or any combination thereof. In one embodiment, one or more carbon atoms may be optionally and independently replaced with one or more heteroatoms such as O, S, N, B, or any combination thereof. In one embodiment, the cycloalkyl group is attached to the parent structure through one or more independent divalent intervening substituent groups. Some examples of cycloalkyl groups, which are not intended to be limiting, include cyclopropyl, cyclobutyl, cyclopentyl, cyclohexyl, cycloheptyl, cyclooctyl, cyclononyl, cyclopentenyl, cyclohexenyl, bicyclo[2.2.1]heptanyl, bicyclo[3.2.1]octanyl and bicyclo[5.2.0]nonanyl, and the like.

In one embodiment, an alkenyl group is a univalent, straight or branched, substituted or unsubstituted, unsaturated hydrocarbon radical. In one embodiment, the alkenyl group has the general formula (notwithstanding optional substitution, higher degree of unsaturation, or the like) —CₙH₂ₙ₋₂. In one embodiment, n is 2-20 ((C₂-C₂₀) alkenyl), which may suitably include C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, and C₂₀ alkenyl groups. In one embodiment, the alkenyl group may be straight or branched, substituted or unsubstituted, have more than one degree of unsaturation, or any combination thereof. In one embodiment, one or more carbon atoms may be optionally and independently replaced with one or more heteroatoms such as O, S, N, B, or any combination thereof. In one embodiment, the alkenyl group is attached to the parent structure through one or more independent divalent intervening substituent groups. Some examples of alkenyl groups, which are not intended to be limiting, include ethenyl, 1-propenyl, 2-propenyl(allyl), iso-propenyl, 2-methyl-1-propenyl, 1-butenyl, 2-butenyl, alkadienes, alkatrienes, and the like.

In one embodiment, an alkynyl group is a univalent, straight or branched, substituted or unsubstituted, hydrocarbon radical that contains one or more carbon-carbon triple bond. In one embodiment, the alkenyl group has the general formula (notwithstanding optional substitution, higher degree of unsaturation, or the like) —CₙH₂ₙ₋₃. In one embodiment, n is 2-20 ((C₂-C₂₀) alkynyl), which may suitably include C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, and C₂₀ alkynyl groups. In one embodiment, the alkynyl group may be straight or branched, substituted or unsubstituted, have more than one degree of unsaturation, or any combination thereof. In one embodiment, one or more carbon atoms may be optionally and independently replaced with one or more heteroatoms such as O, S, N, B, or any combination thereof. In one embodiment, the alkynyl group is attached to the parent structure through one or more independent divalent intervening substituent groups. Some examples of alkynyl groups, which are not intended to be limiting, include alkadiynes, alkatriynes, ethynyl, propynyl, butynyl, and the like.

In one embodiment, an aryl group is a univalent, substituted or unsubstituted, monocyclic or polycyclic aromatic hydrocarbon radical. In one embodiment, an aryl group is a radical which, in accordance with Hückel's theory, includes a cyclic, delocalized (4n+2) pi-electron system. In one embodiment the aryl group is a C₅-C₂₀ aryl group. The C₅-C₂₀ aryl group may suitably include C₅, C₆, C₇, C₈, C₉, C₁₀, C₁₁, C₁₂, C₁₃, C₁₄, C₁₅, C₁₆, C₁₇, C₁₈, C₁₉, and C₂₀ aryl groups. In one embodiment, the aryl group may be substituted or unsubstituted, be substituted with two or more groups that taken together form a cyclic group, or any combination thereof. In one embodiment, the aryl group is attached to the parent structure through one or more independent divalent intervening substituent groups. Some examples of aryl groups, which are not intended to be limiting, include phenyl, naphthyl, tetrahydronaphthyl, phenanthryl, pyrenyl, anthryl, indanyl, chrysyl, and the like.

In one embodiment, a heterocyclic group is a univalent, substituted or unsubstituted, saturated or unsaturated, mono- or polycyclic hydrocarbon radical that contains, one or more heteroatoms in one or more of the rings. In one embodiment, the heterocyclic group is a C₃-C₂₀ cyclic group, in which one or more ring carbons is independently replaced with one or more heteroatoms. The C₃-C₂₀ heterocyclic group may suitably include C₃, C₄, C₅, C₆, C₇, C₈, C₉, C₁₀, C₁₁, C₁₂, C₁₃, C₁₄, C₁₅, C₁₆, C₁₇, C₁₈, C₁₉, and C₂₀ cyclic groups in which one or more ring carbons is independently replaced with one or more heteroatoms. In one embodiment, the heteroatoms are selected from one or more of N, O, or S, or any combination thereof. In one embodiment, the N or S or both may be independently substituted with one or more substituents. In one embodiment, the heterocyclic group is substituted or unsubstituted, saturated or unsaturated, mono-, bi-, tri-, or poly-cyclic, or any combination thereof. In one embodiment, one or more hydrogens may be optionally and independently replaced by one or more substituent groups. In one embodiment, the heterocyclic group may include one or more carbon-carbon double bonds, carbon-carbon triple bonds, carbon-nitrogen double bonds, or any combination thereof. In one embodiment, the heterocyclic group is attached to the parent structure through one or more independent divalent intervening substituent groups. Some examples of heterocyclic groups, which are not intended to be limiting, include azetidinyl, tetrahydrofuranyl, imidazolidinyl, pyrrolidinyl, piperidinyl, piperazinyl, oxazolidinyl, thiazolidinyl, pyrazolidinyl, thiomorpholinyl, tetrahydrothiazinyl, tetrahydrothiadiazinyl, morpholinyl, oxetanyl, tetrahydrodiazinyl, oxazinyl, oxathiazinyl, indolinyl, isoindolinyl, quinuclidinyl, chromanyl, isochromanyl, benzoxazinyl, tetrahydrofuran-2-yl, tetrahydrofuran-3-yl, imidazolidin-1-yl, imidazolidin-2-yl, imidazolidin-4-yl, pyrrolidin-1-yl, pyrrolidin-2-yl, pyrrolidin-3-yl, piperidin-1-yl, piperidin-2-yl, piperidin-3-yl, piperazin-1-yl, piperazin-2-yl, piperazin-3-yl, 1,3-oxazolidin-3-yl, isothiazolidine, 1,3-thiazolidin-3-yl, 1,2-pyrazolidin-2-yl, 1,3-pyrazolidin-1-yl, thiomorpholinyl, 1,2-tetrahydrothiazin-2-yl, 1,3-tetrahydrothiazin-3-yl, tetrahydrothiadiazinyl, morpholinyl, 1,2-tetrahydrodiazin-2-yl, 1,3-tetrahydrodiazin-1-yl, 1,4-oxazin-2-yl, 1,2,5-oxathiazin-4-yl, and the like

In one embodiment, a heteroaryl group is univalent, substituted or unsubstituted, monocyclic or polycyclic aromatic hydrocarbon radical in which one or more ring carbons is independently replaced with one or more heteroatoms selected from O, S and N. In one embodiment, in addition to said heteroatom, the heteroaryl group may optionally have up to 1, 2, 3, or 4 N atoms in the ring. In one embodiment, the heteroaryl group is an aryl group in which one or more ring carbons are independently replaced with one or more heteroatoms. In one embodiment, a heteroaryl group is an aromatic radical, which contains one or more heteroatoms and which, in accordance with Hückel's theory, includes a cyclic, delocalized (4n+2) pi-electron system. In one embodiment, the heteroaryl group is a C₅-C₂₀ heteroaryl group. The C₅-C₂₀ heteroaryl group may suitably include C₅, C₆, C₇, C₈, C₉, C₁₀, C₁₁, C₁₂, C₁₃, C₁₄, C₁₅, C₁₆, C₁₇, C₁₈, C₁₉, and C₂₀ aryl groups in which one or more than one ring carbon is independently replaced with one or more heteroatoms. In one embodiment, the heteroaryl group may be substituted or unsubstituted, be substituted with two or more groups that taken together form a cyclic group, or any combination thereof. In one embodiment, the heteroaryl group is attached to the parent structure through one or more independent divalent intervening substituent groups. Some examples of heteroaryl groups, which are not intended to be limiting, include heteroaryl group includes pyridyl, pyrazinyl, pyrimidinyl, pyridazinyl, thienyl, furyl, imidazolyl, pyrrolyl, oxazolyl (e.g., 1,3-oxazolyl, 1,2-oxazolyl), thiazolyl (e.g., 1,2-thiazolyl, 1,3-thiazolyl), pyrazolyl, tetrazolyl, triazolyl (e.g., 1,2,3-triazolyl, 1,2,4-triazolyl), oxadiazolyl (e.g., 1,2,3-oxadiazolyl), thiadiazolyl (e.g., 1,3,4-thiadiazolyl), quinolyl, isoquinolyl, benzothienyl, benzofuryl, indolyl, and the like.

In one embodiment, an aralkyl group is a univalent radical derived from one or more aryl groups attached to one or more of an alkylene group, cycloalkylene group, alkenylene group, alkynylene group, or combination thereof. The alkylene, cycloalkylene, alkenylene, and alkynylene groups are divalent radicals derived from the removal of hydrogen from the respective alkyl, cycloalkyl, alkenyl, or alkynyl groups. In this context, any combination of aryl group and alkyl, cycloalkyl, alkenyl, or alkynyl group is contemplated. In one embodiment, the aryl group is attached to the parent structure through one or more of the alkylene group, cycloalkylene group, alkenylene group, alkynylene group, or combination thereof as appropriate. In one embodiment, the aralkyl group may be attached to the parent structure through one or more independent divalent intervening substituent groups.

In one embodiment, a heteroaralkyl group is a univalent radical derived from one or more heteroaryl groups attached to one or more of an alkylene group, cycloalkylene group, alkenylene group, alkynylene group, or combination thereof. The alkylene, cycloalkylene, alkenylene, and alkynylene groups are divalent radicals derived from the removal of hydrogen from the respective alkyl, cycloalkyl, alkenyl, or alkynyl groups. In this context, any combination of heteroaryl group and alkyl, cycloalkyl, alkenyl, or alkynyl group is contemplated. In one embodiment, the heteroaryl group is attached to the parent structure through one or more of the alkylene group, cycloalkylene group, alkenylene group, alkynylene group, or combination thereof as appropriate. In one embodiment, the heteroaralkyl group may be attached to the parent structure through one or more independent divalent intervening substituent groups.

In one embodiment, a halo group is a univalent halogen radical or halogen-containing substituent group, e.g., one that is or contains one or more F, Br, Cl, I, or combination thereof. As used herein, the term “halogen” or “halo” includes fluoro, chloro, bromo, or iodo, or fluoride, chloride, bromide or iodide. In one embodiment, a halogen containing substituent group may suitably include a substituent group in which one or more hydrogen atoms are independently replaced with one or more halogens. In one embodiment, the halo group may be attached to the parent structure through one or more independent divalent intervening substituent groups.

In one embodiment, a hydroxy group is a univalent hydroxyl radical (—OH) or hydroxy-containing subsituent group, e.g., one that is or contains one or more —OH. As used herein the term, “hydroxy” includes an —OH group. In one embodiment, a hydroxy-containing subsituent group may suitably include a subsituent group in which one or more hydrogen atoms are independently replaced with one or more —OH groups. In one embodiment, the hydroxyl group may be attached to the parent structure through one or more independent divalent intervening substituent groups.

In one embodiment, an oxo group is a univalent radical that contains an oxygen atom, ═O, doubly bonded to carbon or another element. In one embodiment, the oxo group suitably includes aldehydes, carboxylic acids, ketones, sulfonic acids, amides, esters, and combinations thereof. In one embodiment, the oxo group may be attached to the parent structure through one or more independent divalent intervening substituent groups.

In one embodiment, a mercapto or thiol group is a univalent —SR radical or an —SR— containing group. The R group is suitably chosen from any of the substituent groups. In one embodiment, the mercapto group may be attached to the parent structure through one or more independent divalent intervening substituent groups.

In one embodiment, an amino group is a univalent —NH₂ radical or an —NH₂-containing subsituent group. In one embodiment, the amino group may be attached to the parent structure through one or more independent divalent intervening substituent groups.

In one embodiment, an alkylamino group is a univalent —NRH radical or an —NRH-containing subsituent group. The R group is suitably chosen from any of the substituent groups. In one embodiment, the alkylamino group may be attached to the parent structure through one or more independent divalent intervening substituent groups.

In one embodiment, a dialkylamino group is a univalent —NRR radical or an —NRR-containing subsituent group. The R groups may be the same or different and are suitably and independently chosen from any of the substituent groups. In one embodiment, the dialkylamino group may be attached to the parent structure through one or more independent divalent intervening substituent groups.

In one embodiment, a carbonyl group is a univalent radical that contains a —CR(═O) group. In one embodiment, the carbonyl group suitably includes aldehydes, ketones, and combinations thereof. The R group is suitably chosen from any of the substituent groups. In one embodiment, the carbonyl group may be attached to the parent structure through one or more independent divalent intervening substituent groups.

In one embodiment, a carboxylic acid group is a univalent —C(═O)OH radical or a —C(═O)OH-containing subsituent group. In one embodiment, the carboxylic acid group may be attached to the parent structure through one or more independent divalent intervening substituent groups.

In one embodiment, a carboxylate group is a univalent —C(═O)O⁻ anion, —C(═O)OR, or —C(═O)OM, wherein M is a metal cation, or —C(═O)O⁻ anion, —C(═O)OR, or —C(═O)OM-containing substituent group. The R group is suitably chosen from any of the substituent groups. The metal cation is suitably chosen from Li, Na, K, and the like. In one embodiment, the carboxylate group may be attached to the parent structure through one or more independent divalent intervening substituent groups.

In one embodiment, an amidine group is a univalent —C(═NR)NRR radical or a —C(═NR)NRR-containing substituent group. The R groups may be the same or different and are suitably and independently chosen from any of the substituent groups. In one embodiment, the amidine group may be attached to the parent structure through one or more independent divalent intervening substituent groups.

In one embodiment, an amide group is a univalent -E(═O)NRR radical or a -E(═O)NRR-containing substituent group, in which E may be other than carbon, e.g., a chalcogen (e.g., S, Se, Te), or P. In one embodiment, the amide group suitably includes univalent lactams, peptides, phosphoramides, or sulfamides, —S(═O)₂NRR, —P(═O)(OH)NRR, and the like. The R groups may be the same or different and are suitably and independently chosen from any of the substituent groups. In one embodiment, the amide group may be attached to the parent structure through one or more independent divalent intervening substituent groups.

In one embodiment, a carbamoyl group is a univalent —C(═O)NRR radical or a —C(═O)NRR-containing substituent group. The R groups may be the same or different and are suitably and independently chosen from any of the substituent groups. In one embodiment, the carbamoyl group may be attached to the parent structure through one or more independent divalent intervening substituent groups.

In one embodiment, a sulfonyl group is a univalent —S(═O)₂R radical or a —S(═O)₂R-containing substituent group. The R group is suitably chosen from any of the substituent groups. In one embodiment, the sulfonyl group may be attached to the parent structure through one or more independent divalent intervening substituent groups.

In one embodiment, an alkylthio or sulfide group is a univalent —SR radical or an —SR-containing substituent group. The R group is suitably chosen from any of the substituent groups. In one embodiment, the alkylthio group may be attached to the parent structure through one or more independent divalent intervening substituent groups.

In one embodiment, an alkoxy group is a univalent radical derived from an —O-alkyl group. In one embodiment, the alkylthio group may be attached to the parent structure through one or more independent divalent intervening substituent groups.

In one embodiment, an aryloxy group is a univalent radical derived from an —O-aryl group. In one embodiment, the aryloxy group may be attached to the parent structure through one or more independent divalent intervening substituent groups.

In one embodiment, a heteroaryloxy group is a univalent radical derived from an —O-heteroaryl group. In one embodiment, the heteroaryloxy group may be attached to the parent structure through one or more independent divalent intervening substituent groups.

In one embodiment, an aralkoxy group is a univalent radical derived from an —O-aralkyl group. In one embodiment, the aralkoxy group may be attached to the parent structure through one or more independent divalent intervening substituent groups.

In one embodiment, a heteroaralkoxy group is a univalent radical derived from an —O-heteroaryl group. In one embodiment, the heteroaralkoxy group may be attached to the parent structure through one or more independent divalent intervening substituent groups.

In one embodiment, an alkylcarbonyl group is a univalent is radical derived from a -carbonyl-alkyl group. In one embodiment, the alkylcarbonyl group may be attached to the parent structure through one or more independent divalent intervening substituent groups.

In one embodiment, an alkoxycarbonyl group is a univalent radical derived from a -carbonyl-O-alkyl group. In one embodiment, the alkoxycarbonyl group may be attached to the parent structure through one or more independent divalent intervening substituent groups.

In one embodiment, an alkylaminocarbonyl group is a univalent radical derived from a -carbonyl-alkylamino group. In one embodiment, the heteroaralkoxy group may be attached to the parent structure through one or more independent divalent intervening substituent groups.

In one embodiment, a dialkylamino carbonyl group is a univalent radical derived from a -carbonyl-dialkylamino group. In one embodiment, the dialkylamino carbonyl group may be attached to the parent structure through one or more independent divalent intervening substituent groups.

In one embodiment, an arylcarbonyl group is a univalent radical derived from a -carbonyl-aryl group. In one embodiment, the arylcarbonyl group may be attached to the parent structure through one or more independent divalent intervening substituent groups.

In one embodiment, an aryloxycarbonyl group is a univalent radical derived from a -carbonyl-O-aryl group. In one embodiment, the aryloxycarbonyl group may be attached to the parent structure through one or more independent divalent intervening substituent groups.

In one embodiment, an alkylsulfonyl group is a univalent radical derived from a -sulfonyl-alkyl group. In one embodiment, the alkylsulfonyl group may be attached to the parent structure through one or more independent divalent intervening substituent groups.

In one embodiment, an arylsulfonyl group is a univalent radical derived from a -sulfonyl-aryl group. In one embodiment, the arylsulfonyl group may be attached to the parent structure through one or more independent divalent intervening substituent groups.

In one embodiment, a perhaloalkyl group is a univalent radical derived from a completely or substantially completely halogenated alkyl group. In one embodiment, the parhaloalkyl group may be attached to the parent structure through one or more independent divalent intervening substituent groups.

In one embodiment, a perhaloalkoxy group is a univalent radical derived from a completely or substantially completely halogenated alkoxy group. In one embodiment, the arylsulfonyl group may be attached to the parent structure through one or more independent divalent intervening substituent groups.

In one embodiment, a perhalocycloalkyl group is a univalent radical derived from a completely or substantially completely halogenated cycloalkyl group. In one embodiment, the perhalocycloalkyl group may be attached to the parent structure through one or more independent divalent intervening substituent groups.

In one embodiment, a perhaloalkenyl group is a univalent radical derived from a completely or substantially completely halogenated alkenyl group. In one embodiment, the perhaloalkenyl group may be attached to the parent structure through one or more independent divalent intervening substituent groups.

In one embodiment, a perhaloalkynyl group is a univalent radical derived from a completely or substantially completely halogenated alkynyl group. In one embodiment, the perhaloalkynyl group may be attached to the parent structure through one or more independent divalent intervening substituent groups.

In one embodiment, a perhaloaryl group is a univalent radical derived from a completely or substantially completely halogenated aryl group. In one embodiment, the perhaloaryl group may be attached to the parent structure through one or more independent divalent intervening substituent groups.

In one embodiment, a perhaloaralkyl group is a univalent radical derived from a completely or substantially completely halogenated aralkyl group. In one embodiment, the perhaloaralkyl group may be attached to the parent structure through one or more independent divalent intervening substituent groups.

In one embodiment, an alkylcarbonyloxy group is a univalent radical derived from an —O-carbonyl-alkyl group. In one embodiment, the alkylcarbonyloxy group may be attached to the parent structure through one or more independent divalent intervening substituent groups.

In one embodiment, an alkoxycarbonyloxy group is a univalent radical derived from an —O-carbonyl-O-alkyl group. In one embodiment, the alkoxycarbonyloxy group may be attached to the parent structure through one or more independent divalent intervening substituent groups.

In one embodiment, an alkylsulfonyloxy group is a univalent radical derived from an —O-sulfonyl-alkyl group. In one embodiment, the alkylsulfonyloxy group may be attached to the parent structure through one or more independent divalent intervening substituent groups.

In one embodiment, an alkoxysulfonyloxy group is a univalent radical derived from an —O-sulfonyl-O-alkyl group. In one embodiment, the alkoxysulfonyloxy group may be attached to the parent structure through one or more independent divalent intervening substituent groups.

In one embodiment, an arylcarbonyloxy group is a univalent radical derived from an —O-carbonyl-aryl group. In one embodiment, the arylcarbonyloxy group may be attached to the parent structure through one or more independent divalent intervening substituent groups.

In one embodiment, an aryloxycarbonyloxy group is a univalent radical derived from an —O-carbonyl-O-aryl group group. In one embodiment, the aryloxycarbonyloxy group may be attached to the parent structure through one or more independent divalent intervening substituent groups.

In one embodiment, an arylsulfonyloxy group is a univalent radical derived from an —O-sulfonyl-aryl group. In one embodiment, the arylsulfonyloxy group may be attached to the parent structure through one or more independent divalent intervening substituent groups.

In one embodiment, an aryloxysulfonyloxy group is a univalent radical derived from an —O-sulfonyl-O-aryl group. In one embodiment, the aryloxysulfonyloxy group may be attached to the parent structure through one or more independent divalent intervening substituent groups.

In one embodiment, referring to two groups taken together to form a cyclic group, the cyclic group may be suitably derived from a divalent cycloalkylene group or divalent heterocyclic group. The divalent cycloalkylene and heterocyclic groups may be suitably derived from the respective cycloalkyl or heterocyclic groups.

In one embodiment, referring to two groups taken together to form an aryl group, the aryl group may be suitably derived from a divalent arylene group or divalent heteroarlyene group. The divalent arylene and heteroarylene groups may be suitably derived from the respective aryl or heteroaryl groups.

In one embodiment, referring to the replacement of one or more than one atom in each group with one or more heteroatoms, the heteroatoms may be suitably chosen from N, O, P, S, B, or any combination thereof as appropriate.

In one embodiment, the structure

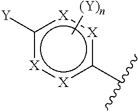

may have one of the following three structures:

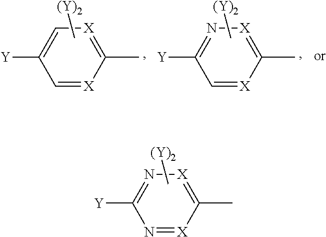

wherein each X is independently carbon or nitrogen, and wherein X is carbon, it independently comprises a Y substituent. In the three structures shown above, in one embodiment, the X's may be carbon, each carbon independently comprising a Y substituent.

In one embodiment, the structure

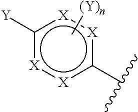

may have one of the following structures:

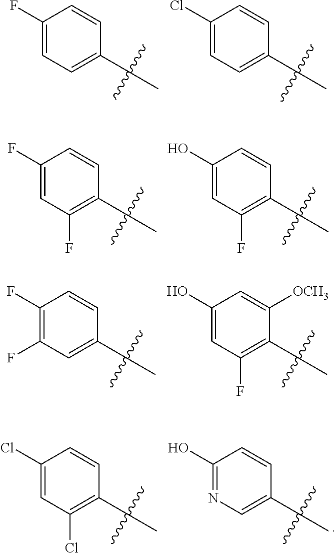

In one embodiment, Y may be an alkyl group, a cycloalkyl group, a halo group, a perfluoroalkyl group, a perfluoroalkoxy group, an alkenyl group, an alkynyl group, a hydroxy group, an oxo group, a mercapto group, an alkylthio group, an alkoxy group, an aryl group, a heteraryl group, an aryloxy group, a heteroaryloxy group, an aralkyl group, a heteroaralkyl group, an aralkoxy group, a heteroaralkoxy group, an HO—(C═O)— group, an amino group, an alkylamino group, a dialkylamino group, a carbamoyl group, an alkylcarbonyl group, an alkoxycarbonyl group, an alkylaminocarbonyl group, a dialkylamino carbonyl group, an arylcarbonyl group, an aryloxycarbonyl group, an alkylsulfonyl group, or an arylsulfonyl group, or have the following structure:

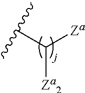

in which each Z^{a} is independently either hydrogen, hydroxyl, halogen, or a substituent group; and

“j” is independently either zero or an integer from one to four.

In one embodiment, the structure:

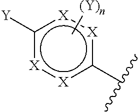

has the following structure:

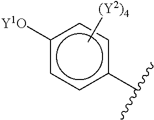

in which each Y¹ is independently a hydrogen or (C₁-C₆)alkyl; and

each Y² is independently a Y¹, hydroxyl group, halo group, —N₃, —CN, —SH, or —N(Y¹)₂.

In one embodiment, the subject compound has the following structure:

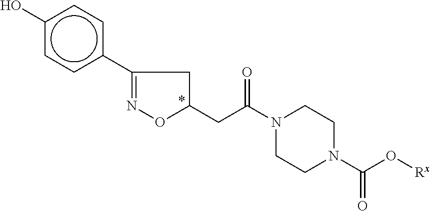

wherein R^{X} is a (C₁-C₆)alkyl, (C₂-C₆)alkenyl, (C₂-C₆)alkynyl, phenyl, (C₅-C₁₄)aryl, (C₄-C₁₄)heteroaryl, (C₂-C₁₄)heterocyclic or (C₃-C₁₀)cycloalkyl group.

In one embodiment, the subject compound has the following structure:

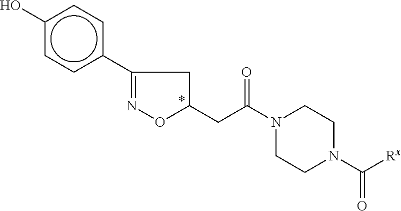

wherein R^{X} is a (C₁-C₆)alkyl, (C₂-C₆)alkenyl, (C₂-C₆)alkynyl, phenyl, (C₅-C₁₄)aryl, (C₄-C₁₄)heteroaryl, (C₂-C₁₄)heterocyclic or (C₃-C₁₀)cycloalkyl group.

In one embodiment, the subject compound has the following structure:

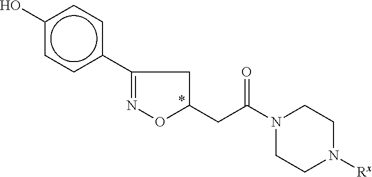

wherein R^{X} is a sulfonyl, carbonyl, (C₁-C₆)alkylsulfonyl, (C₁-C₆)alkylcarbonyl, (C₅-C₁₄)arylsulfonyl, (C₅-C₁₄)arylcarbonyl, (C₁-C₆)alkyl, (C₂-C₆)alkenyl, (C₂-C₆)alkynyl, phenyl, (C₅-C₁₄)aryl, (C₄-C₁₄)heteroaryl, (C₂-C₁₄)heterocyclic or (C₃-C₁₀)cycloalkyl group.

In one embodiment, the subject compound has the following structure:

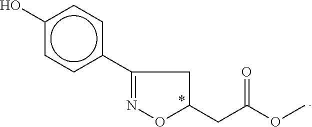

In one embodiment, the subject compound has the following structure:

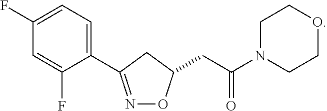 [SMILES: O=C(C[C@H]1CC(c2ccc(F)cc2F)=NO1)N1CCOCC1]

In one embodiment, the subject compound is an ester of (R)-3-(4-hydroxyphenyl)-4,5-dihydro-5-isoxazolineacetic acid. In another embodiment, the ester is the methyl ester thereof (sometimes identified as “ISO-1”).

In one embodiment, the subject compound is an ester of 2-{3-(4-hydroxy-phenyl)-4,5-dihydro-isoxazol-5-yl}-3-phenyl-propanoic acid. In another embodiment, the ester is the methyl ester thereof (sometimes identified as “ISO-2”).

In one embodiment, the subsituent groups described herein may be suitably and independently chosen from one or more of a hydrogen, an azido group, a carbamido group, a carbazoyl group, a cyanato group, a cyano group, an isocyanato group, an isocyano group, a hydroxamino group, a guanidino group, a guanyl group, an imino group, a nitro group, a phospho group, a phosphate group, a phosphine group, a sulfo group, a sulfate group, a sulfonyl group, a carbonyl group, a carboxylic acid group, a carboxylate group, an alkyl group, a cycloalkyl group, a halo group, an alkenyl group, an alkynyl group, a hydroxy group, an oxo group, a mercapto group, an alkylthio group, an alkoxy group, an aryl group, a heterocyclic group, a heteroaryl group, an aryloxy group, a heteroaryloxy group, an aralkyl group, a heteroaralkyl group, an aralkoxy group, a heteroaralkoxy group, an amino group, an alkylamino group, a dialkylamino group, an amidine group, an amide group, a carbamoyl group, an alkylcarbonyl group, an alkoxycarbonyl group, an alkylaminocarbonyl group, a dialkylamino carbonyl group, an arylcarbonyl group, an aryloxycarbonyl group, an alkylsulfonyl group, an arylsulfonyl group, an alkylcarbonyloxy group, an alkoxycarbonyloxy group, an alkylsulfonyloxy group, an alkoxysulfonyloxy group, an arylcarbonyloxy group, an aryloxycarbonyloxy group, an arylsulfonyloxy group, an aryloxysulfonyloxy group, an a perhaloalkyl group, a perhaloalkoxy group, a perhalocycloalkyl group, a perhaloalkenyl group, a perhaloalkynyl group, a perhaloaryl group, a perhaloaralkyl group, or combination thereof. Univalent residues or divalent intervening residues of any substituent group or combination thereof may be suitably used as appropriate.

In one embodiment, the divalent intervening subsituent groups may be suitably and independently chosen from one or more of an azo group, an azino group, an azoxy group, a carbonyl group, a dioyl group, a diazoamino group, a disulfinyl group, a dithio group, an oxy group, a hydrazo group, an oxalyl group, a sulfonyl group, a a thiocarbonyl group, a thionyl group, a phosphono ester group, a carboxylate group, a thio group; divalent residues of one or more of the following groups: an alkyl group, a cycloalkyl group, an alkenyl group, an alkynyl group, an alkylthio group, an alkyloxy group, an aryl group, a heterocyclic group, a heteroaryl group, an aryloxy group, a heteroaryloxy group, an aralkyl group, a heteroaralkyl group, an aralkoxy group, a heteroaralkoxy group, an amino group, an alkylamino group, a dialkylamino group, an amidine group, an amide group, a carbamoyl group, an alkylcarbonyl group, an alkoxycarbonyl group, an alkylaminocarbonyl group, a dialkylamino carbonyl group, an arylcarbonyl group, an aryloxycarbonyl group, an alkylsulfonyl group, an arylsulfonyl group, an alkylcarbonyloxy group, an alkoxycarbonyloxy group, an alkylsulfonyloxy group, an alkoxysulfonyloxy group, an arylcarbonyloxy group, an aryloxycarbonyloxy group, an arylsulfonyloxy group, an aryloxysulfonyloxy group, an a perhaloalkyl group, a perhaloalkoxy group, a perhalocycloalkyl group, a perhaloalkenyl group, a perhaloalkynyl group, a perhaloaryl group, a perhaloaralkyl group, combination thereof; or combination thereof.

In one embodiment, the subject compound, or salt thereof, or prodrug thereof, or combination thereof, may be administered alone.

In one embodiment, the subject compound, or salt thereof, or prodrug thereof, or combination thereof, may be administered in combination with at least one pharmaceutically acceptable carrier, in the form of a pharmaceutical composition.

In one embodiment, the subject compound is in the form of the metabolite, isotopically-labeled, tautomer, isomer, and/or atropisomer.

Non-limiting examples of some techniques for the formulation and administration of the subject compound, or salt thereof, or prodrug thereof, or combination thereof may be found in Remington's Pharmaceutical Sciences, Mack Publishing Co., Easton, Pa., latest addition.

Suitable routes of administration may, for example, include oral, rectal, transmucosal, buccal, intravaginal, or intestinal administration; parenteral delivery, including intramuscular, subcutaneous, intramedullary injections, as well as intrathecal, direct intraventricular, intravenous, intraperitoneal, intranasal, or intraocular injections, and optionally in a depot or sustained release formulation. Furthermore, one may administer the subject compound in a targeted drug delivery system, for example in a liposome.

The pharmaceutical composition may be manufactured in a manner that is itself known, e.g., by means of conventional mixing, dissolving, dragee-making, levitating, emulsifying, encapsulating, entrapping, or lyophilizing processes. The pharmaceutical compositions thus may be formulated in conventional manner using one or more physiologically acceptable carriers comprising excipients and auxiliaries that facilitate processing of the subject compound into preparations, which can be used pharmaceutically. Proper formulation may be dependent upon the route of administration chosen.

For injection, the subject compound, salt thereof, prodrug thereof, or combination thereof may be formulated in aqueous solutions, e.g., in physiologically compatible buffers, such as Hank's solution, Ringer's solution, or physiological saline buffer. For transmucosal administration, penetrants appropriate to the barrier to be permeated may be used. Such penetrants are known in the art.

For oral administration, the subject compound, salt thereof, prodrug thereof, or combination thereof may be formulated by combining the subject compound, salt thereof, prodrug thereof, or combination thereof with pharmaceutically acceptable carriers known to those in the art. Such carriers may be suitably used to formulate the subject compound, salt thereof, prodrug thereof, or combination thereof as tablets, pills, dragees, capsules, liquids, gels, syrups, slurries, suspensions and the like, for oral ingestion by the subject to be treated. Pharmaceutical preparations for oral use can be obtained by combining the subject compound, salt thereof, prodrug thereof, or combination thereof with a solid excipient, optionally grinding the resulting mixture, and processing the mixture of granules, after adding suitable auxiliaries, if desired, to obtain tablets or dragee cores. Suitable excipients may include, for example, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate.

Dragee cores may be provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of doses.

Other pharmaceutical preparations that can be used orally include push-fit capsules made of gelatin, as well as soft, sealed capsules made of gelatin and a plasticizer, such as glycerol or sorbitol. The push-fit capsules can contain the subject compound, salt thereof, prodrug thereof, or combination thereof in admixture with filler such as lactose, binders such as starches, and/or lubricants such as talc or magnesium stearate and, optionally, stabilizers. In soft capsules, the subject compound, salt thereof, prodrug thereof, or combination thereof may be dissolved or suspended in suitable liquids, such as fatty oils, liquid paraffin, or liquid polyethylene glycols. One or more stabilizers may be added if desired. The formulations for oral administration may be in dosages suitable for such administration.

For buccal administration, the compositions may take the form of tablets or lozenges formulated in conventional manner.

For administration by inhalation, the subject compound, salt thereof, prodrug thereof, or combination thereof may be delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g., gelatin for use in an inhaler or insufflator may be formulated containing a powder mix of the compound, salt thereof, prodrug thereof, or combination thereof and a suitable powder base such as lactose or starch.

The subject compound, salt thereof, prodrug thereof, or combination thereof may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

Pharmaceutical formulations for parenteral administration may include aqueous solutions of the subject compound, salt thereof, prodrug thereof, or combination thereof in water-soluble form. Suspensions of the subject compound, salt thereof, prodrug thereof, or combination thereof may be prepared as appropriate oily injection suspensions. Non-limiting examples of lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as polyionic block (co)polymer, sodium carboxymethyl cellulose, sorbitol, or dextran. Optionally, the suspension may also contain suitable stabilizers or agents which increase the solubility of the subject compound, salt thereof, prodrug thereof, or combination thereof to allow for the preparation of highly concentrated solutions, e.g., polyionic block (co)polymers.

The subject compound, salt thereof, prodrug thereof, or combination thereof may be in powder form for constitution with a suitable vehicle, e.g., sterile pyrogen-free water, before use.

The subject compound, salt thereof, prodrug thereof, or combination thereof may also be formulated in rectal compositions such as suppositories or retention enemas, e.g., containing conventional suppository bases such as cocoa butter or other glycerides.

The subject compound, salt thereof, prodrug thereof, or combination thereof may also be formulated as a depot preparation. Such long acting formulations may be administered by implantation (for example subcutaneously or intramuscularly) or by intramuscular injection. Thus, for example, the subject compound, salt thereof, prodrug thereof, or combination thereof may be formulated with suitable polymeric or hydrophobic materials (for example as an emulsion in an acceptable oil) or ion exchange resins, or as sparingly soluble derivatives, for example, as a sparingly soluble salt.

The subject compound, salt thereof, prodrug thereof, or combination thereof may be formulated into delivery vehicles or carriers such as liposomes and emulsions. If desired, organic solvents such as dimethylsulfoxide also may be employed for formulating into the liposome or emulsion. If desired, the subject compound, salt thereof, prodrug thereof, or combination thereof may be delivered using a sustained-release system, such as semipermeable matrices of solid hydrophobic polymers containing the therapeutic agent. Sustained-release materials are known in the art. Sustained-release capsules may, depending on their chemical nature, release the subject compound, salt thereof, prodrug thereof, or combination thereof for a few weeks up to over 100 days.

The pharmaceutical compositions also may comprise suitable solid- or gel-phase carriers or excipients. Examples of such carriers or excipients include but are not limited to calcium carbonate, calcium phosphate, various sugars, starches, cellulose derivatives, gelatin, and polymers such as polyethylene glycols.

The subject compound, prodrug thereof, or combination thereof may be provided as salts with pharmaceutically compatible counterions. Pharmaceutically compatible salts may be formed with many acids, including but not limited to hydrochloric, sulfuric, acetic, lactic, tartaric, malic, succinic, etc.; or bases. Salts tend to be more soluble in aqueous or other protonic solvents than are the corresponding free base forms. Non-limiting examples of pharmaceutically acceptable salts, carriers or excipients are well known to those skilled in the art and can be found, for example, in Remington's Pharmaceutical Sciences, 18th Edition, A. R. Gennaro, Ed., Mack Publishing Co., Easton, Pa. (1990). Such salts include, but are not limited to, sodium, potassium, lithium, calcium, magnesium, iron, zinc, hydrochloride, hydrobromide, hydroiodide, acetate, citrate, tartrate and maleate salts, and the like.

In one embodiment, the subject compound, salt thereof, prodrug thereof, or combination thereof, or the pharmaceutical composition that contains the subject compound, salt thereof, prodrug thereof, or combination thereof is used in an effective amount. In one embodiment, the term, “effective amount” means an amount sufficient to reverse, alleviate, or inhibit the progress of the disorder or condition or one or more symptoms of the disorder or condition, or to cause any observable or measurable difference or improvement of the disorder or condition or one or more symptoms of the disorder or condition. In one embodiment, the term, “effective amount” means an amount sufficient to reduce or prevent an increase in the level of resistin in a subject or an amount sufficient to reduce or prevent an increase in the blood glucose level of a subject.

The term “treating” as used herein refers to reversing, alleviating, or inhibiting the progress of the disorder or condition or one or more symptoms of the disorder or condition, or to cause any observable or measurable difference or improvement of the disorder or condition or one or more symptoms of the disorder or condition. The term “treatment” as used herein refers to the act of treating. In one embodiment, the t subject is a mammalian subject, for example, a human subject. In one embodiment, the subject is a human subject in need of treatment.

The terms, “reducing” or “preventing an increase in” as used herein refers to reducing or preventing an increase in the level of resistin or the blood glucose level of a subject suffering from diabetes or at risk of contracting diabetes. The terms, “reduction” or “prevention” as used herein refers to the act of reducing or preventing. In one embodiment, the subject is a mammalian subject, for example, a human subject. In one embodiment, the subject is a human subject in need of reduction or prevention.

In one embodiment, toxicity and therapeutic efficacy may if desired be determined by standard pharmaceutical, pharmacological, and toxicological procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio between LD₅₀ and ED₅₀. In one embodiment, subject compounds, salts thereof, prodrugs thereof, or combinations thereof that exhibit high therapeutic indices (ED₅₀>LD₅₀ or ED₅₀>>LD₅₀) may be used. In one embodiment, the dosage may lie within a range of circulating concentrations that include the ED₅₀ with little or no toxicity. The dosage may vary within this range depending upon the dosage form employed and the route of administration utilized. The exact formulation, route of administration and dosage can be chosen by the individual physician in view of the patient's condition. See, for example, Fingl, et al. (1975), in The Pharmacological Basis of Therapeutics.

Dosage amount and interval may be adjusted individually to provide plasma levels of the subject compound, salt thereof, prodrug thereof, or combination thereof, which are sufficient to establish and/or maintain the desired effects. In one embodiment, the subject compound, salt thereof, prodrug thereof, or combination thereof may be present in a pharmaceutical composition in an amount ranging from 0.1 to 99.9% by weight. These ranges include 0.1, 0.5, 0.9, 1, 2, 3, 4, 5, 6, 7, 8, 9, 10, 20, 30, 40, 50, 60, 70, 80, 90, 99, 99.5, 99.9% by weight and any combination thereof.

In cases of local administration for example, direct introduction into a target organ or tissue, or selective uptake, the effective local concentration of the subject compound, salt thereof, prodrug thereof, or combination thereof, may not be related to plasma concentration.

The amount administered may depend upon the subject being treated, on the subject's weight, on the subject's age, on the severity of the affliction, on the manner of administration, and on the judgment of the prescribing physician.

The compounds or compositions may, if desired, be presented in a pack or dispenser device that may contain one or more unit dosage forms containing the subject compound, salt thereof, prodrug thereof, or combination thereof. The pack may for example comprise metal or plastic foil, such as a blister pack. The pack or dispenser device may be accompanied by instructions for administration. Compositions comprising a compound of the invention formulated in a compatible pharmaceutical carrier may also be prepared, placed in an appropriate container, and labeled for treatment of an indicated condition.

The subject compound, salt thereof, prodrug thereof, or combination thereof can be used in the manufacture of a medicament for the treatment of the disorders and/or symptoms thereof described herein.

In one embodiment, a method for reducing or preventing an increase in the level of resistin in a subject comprises administering to said subject a compound having the following formula:

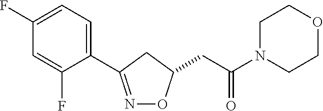 [SMILES: O=C(C[C@H]1CC(c2ccc(F)cc2F)=NO1)N1CCOCC1]

or salt thereof, prodrug thereof, or combination thereof, optionally in contact with one or more pharmaceutical carrier.

In one embodiment, a method for treating diabetes in a subject comprises administering to said subject a compound having the following formula:

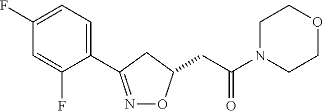 [SMILES: O=C(C[C@H]1CC(c2ccc(F)cc2F)=NO1)N1CCOCC1]

or salt thereof, prodrug thereof, or combination thereof, optionally in contact with one or more pharmaceutical carrier.

In one embodiment, a method for reducing the blood glucose level of a subject suffering from diabetes comprises administering to said subject a compound having the following formula:

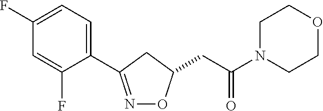 [SMILES: O=C(C[C@H]1CC(c2ccc(F)cc2F)=NO1)N1CCOCC1]

or salt thereof, prodrug thereof, or combination thereof, optionally in contact with one or more pharmaceutical carrier.

In one embodiment, the subject suffers from diabetes.

In one embodiment, the diabetes is type 2 diabetes.

### EXAMPLES

A further understanding can be obtained by reference to certain specific examples, which are provided herein for purposes of illustration only and are not intended to be limiting unless otherwise specified.

The present inventors have found that macrophage migration inhibitory factor (MIF) is a therapeutic target in treatment of non-insulin dependent diabetes mellitus (NIDDM). Macrophage migration inhibitory factor (MIF) is a pro-inflammatory cytokine involved in the pathogenesis of a variety of autoimmune inflammatory diseases. Here, the present inventors have investigated the role of MIF in pathogenesis of non-insulindependent diabetes mellitus (NIDDM) using MIF−/− mice and a mouse model of STZ-induced NIDDM. Following single injection of streptozocin (“STZ”), WT BALB/c mice showed a significant increase in blood glucose levels, developed polyuria and succumbed to disease. In contrast, no such increase in blood glucose was observed in MIF−/− BALB/c mice treated with STZ. These mice produced significantly less inflammatory cytokines and resistin as compared WT mice and failed to develop clinical disease. Finally, oral administration of a small molecule MIF antagonist, CSP1306 to out bred ICR mice following induction of NIDDM significantly lowered blood glucose levels in majority of animals, which was also associated with a significant reduction in levels of pro-inflammatory cytokines IL-6 and TNF-α in the sera. Taken together, these results demonstrate that MIF is involved in pathogenesis of NIDDM and is a therapeutic target to treat this disease.

The present inventors examined the role of MIF in pathogenesis of NIDDM using MIF−/− BALB/c mice which are rendered diabetic by single injection of a subdiabetogenic dose of STZ and then determine whether a small molecule MIF antagonist CSP1306, which is orally bio-available, can be used in treatment of this disease. The results show that MIF plays a critical role in pathogenesis of STZ-induced NIDDM in BALB/c mice. More importantly, the data demonstrate that MIF antagonist, CSPI-1306 is highly effective in suppressing pro-inflammatory cytokine production and in controlling blood sugar levels in diabetic ICR mice when administered orally. These findings show that MIF is a therapeutic target in NIDDM.

Materials and Methods:

Animals

Six to eight weeks old female BALB/c mice and ICR mice were purchased from Harlan and were maintained in a pathogen free environment at animal facilities in accordance with Institutional guidelines. MIF−/− mice were developed as described (12) and backcrossed for more than 10 generations to a BALB/c genetic background.

Induction of Type II Diabetes

ICR, MIF−/− BALB/c and BALB/c mice were fasted 20 hr before induction of diabetes with a single i.p. injection of STZ 90 mg/Kg (for ICR mice) or 130 mg/kg (for BALB/c mice) (Sigma, St. Louis, Mo., U.S.A.) freshly dissolved in 0.05 M citrate buffer, pH 4.5 following the protocol previously reported (13;14). Normal mice of each strain were injected with equivalent volume of citrate buffer as negative controls.

Analysis of Serum Glucose, Body Weight, Urine Volume and Food Consumption

Blood samples from WT and MIF−/− mice were collected at 0, 1, 2, 4, 6, 8, and 10 weeks after STZ injection by tail snipping. Serum glucose levels were determined using Accu-Chek® glucometer. Body weights were measured immediately before blood collection. The animals were kept in individual metabolic cages for 24 hrs and provided with drinking water (100 ml) and food (75 g). The water and food consumption as well as urine output over a 24 hr period was determined. This process was repeated every week until 10 weeks after STZ induction.

Glucose Tolerant Test

Oral glucose tolerance test was carried out in WT and MIF−/− mice at 4 weeks after STZ administration. Animals were fasted for 8 h and then given 2 g/kg glucose solution orally. Blood samples were collected by tail snipping at 30, 60, 90 and 120 minutes after glucose administration and glucose levels were measured as described above.

Analysis of Cytokine, Insulin and Resistin Production In Vivo

Following administration of STZ, blood was collected at 1-week intervals by tail snips. Levels of TNF-α, IL-1β, IL-6, IL-4, IL-10, resistin (all from Peprotech, Mexico), and insulin (Lincon, St. Charles, Mo.) in sera were determined by ELISA as per the manufacturer's instructions.

Islet Culture and mRNA Analysis

Animals were euthanized and pancreatic islets were isolated by collagenase digestion and discontinuous Ficoll-density gradient (Sigma; St. Louis, Mo., USA). After isolation, islets were collected manually and total RNA was extracted with TRIzol (Invitrogen; Carlsbad, Calif., USA). RNA concentration was determined by absorbance at 260 nm, and its integrity was confirmed by electrophoresis on 1% denaturing agarose gel. Single-stranded cDNA was synthesized from 0.5 μg of total RNA by reverse-transcription reaction with 500 units of M-MVL RT (Invitrogen, Carlsbad, Calif., USA). Insulin, glucokinase, GLUT2 and PDX-1 relative expression was evaluated in PCR amplification using the following primers. Insulin: 5′-ATTGTTCCAACATGGCCCTGT-3′ and 5′-TTGCAGTAGTTCTCCAGTTGG-3′; Glucokinase: 5′-GCTTCACCTTCTCCTTCC-3′ and 5′-CCCATATACTTCCCACCGA-3′; GLUT2: 5′-TCACACCAGCATACACAACA-3′ and 5′-TACACTTCGTCCAGCAATGA-3′; PDX-1: 5′-CTCGCTGGGAACGCTGGAACA-3′ and 5′-GCTTTGGTGGATTTCATCCACGG-3′. The amplification was accomplished by incubating 1 μl of the resulting cDNAin a 30 μl reaction volume (50 mmol/l KCl, 150 mmol/l MgCl2, 10 mmol/l Tris-HCl, pH 9.0) containing 100 μmol of specific sense and antisense primers and 0.3 μl of Taq polymerase (Perkin-Elmer). The samples were analyzed in agarose gels by duplicate and corrected for the 18S ribosomal subunit used as internal standard.

Histopathology

Pancreas from all groups were fixed overnight in formaldehyde and embedded in paraffin blocks, after which 5-μm-thick transverse sections were mounted on slides and subsequently stained with hematoxylin-eosin. Using an Olympus BX51 microscope (Olympus American, Melville, N.Y.) equipped with a digital video camera, individual Langerhans Islets were evaluated per sample.

Effect of Orally Administered MIF Antagonist CSP1306 on Diabetes in ICR Mice

Age and sex-matched ICR mice were fasted for 20 hrs and then injected i.p. with STZ (90 mg/Kg). Six-hours later following STZ injection mice were administered CSP1306 or PBS daily in a single dose orally for 30 days. Blood was collected by tail snipping once every week to measure glucose levels using Accu-Chek® glucometer and to determine cytokine levels by ELISA as described above. CPSI 1306 has the following formula:

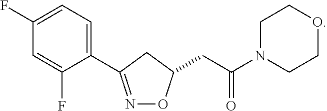 [SMILES: O=C(C[C@H]1CC(c2ccc(F)cc2F)=NO1)N1CCOCC1]

Analysis of Serum Glucose, MIF, TNF-α and Resistin Levels in Patients with NIDDM

Blood was collected by venipunture from NIDDM patients (Males n=27; Females n=46) who came to UNAM. Iztacala Medical Clinic for routine follow-up after an informed consent. Glucose levels were determined by biochemical analysis and serum MIF (R and D Systems, MN), TNF-α (PeproTech, Mexico) and resistin levels were determined by ELISA.

Statistical Analysis:

Comparisons between wild-type MIF+/+ and MIF−/− groups considered in this work were made using student's unpaired t test. A value of p<0.05 was considered significant.

Results

MIF−/− Mice Develop Significantly Less Severe STZ-Induced Type II Diabetes than WT Mice.

To examine the role of MIF in pathogenesis of NIDDM, WT and MIF−/− BALB/c mice were injected i.p. with a single dose of STZ (130 mg/Kg), as described previously (13-15). This subdiabetogenic dose of STZ induces NIDDM which is characterized by progressive increase in blood glucose levels associated with normal non-fasting serum insulin levels (13-15).

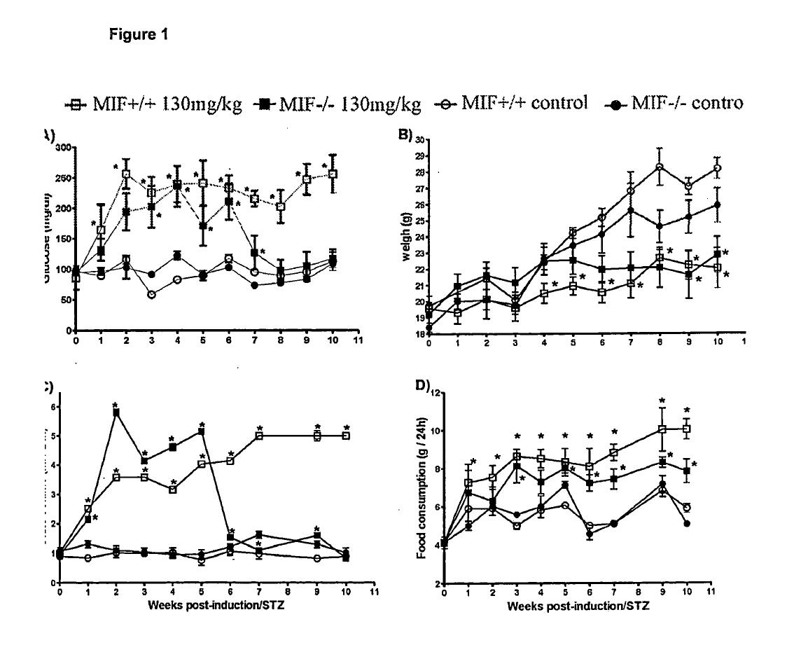

Following development of NIDDM in experimental mice, severity of disease was compared by monitoring blood glucose levels, weight loss and polyuria. Both WT and MIF−/− mice showed a comparable spike in blood glucose levels at week 1 following STZ injection. However, blood glucose levels continued to rise in WT mice and this was associated with the development of severe polyuria, increased food intake and progressive weight loss (FIG. 1A-1D). In contrast, MIF−/− mice showed a drop in their blood glucose levels by week 7, developed minimal polyuria and weight loss (FIG. 1A-1D). Taken together, these results indicate that MIF is involved in pathogenesis of NIDDM.

MIF−/− Mice have Better Glucose Tolerance than WT Mice.

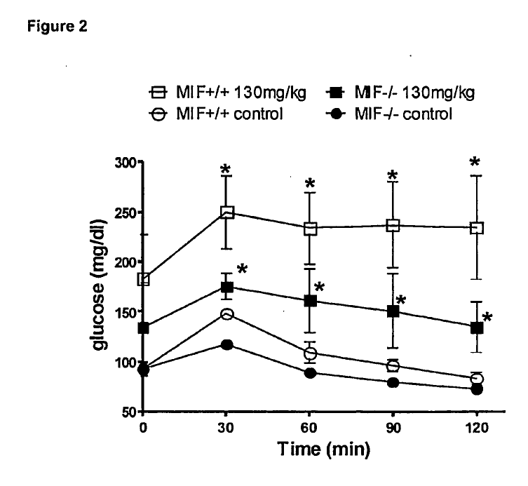

Next, a glucose tolerance test was performed in WT and MIF−/− mice at week 1 following injection with STZ. Following oral administration glucose, both WT and MIF−/− showed a rise in their blood glucose levels (FIG. 2). However, MIF−/− mice lowered their blood glucose by 2 hrs. In contrast, no such drop in serum glucose levels was observed in WT mice, which showed a further spike in their blood glucose (FIG. 2). Non-diabetic WT and MIF−/− displayed comparable glucose tolerance. These data demonstrate that MIF−/− have better glucose tolerance than WT mice.

MIF−/− Mice Produce Significantly Less Inflammatory Cytokines than WT Mice.

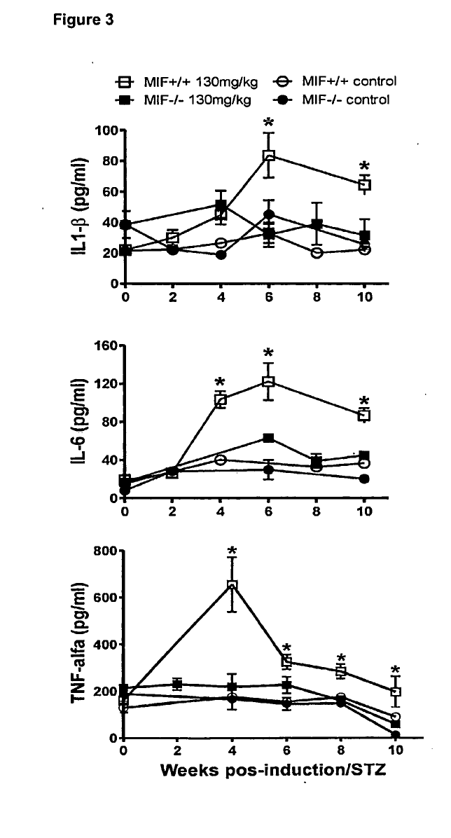

MIF promotes the production of inflammatory cytokines such as IL-1β, IL-6 and TNF-α. Because these cytokines are implicated in exacerbation of NIDDM, these cytokines were measured in sera from diabetic WT and MIF−/− mice by ELISA. Throughout the course of disease, MIF−/− mice produced significantly less IL-6 and TNF-α as compared to WT mice (FIGS. 3A and 2B). Serum levels of IL-1β were comparable in both groups (FIG. 3C). These findings suggest that MIF exacerbates NIDDM at least in part by enhancing production of pro-inflammatory cytokines.

Histopathology of Pancreatic Islets and Quantification of Insulin and Glucose Transporter 2 (GLUT2) mRNA Levels in Islet Cells from MIF−/− and WT Mice.

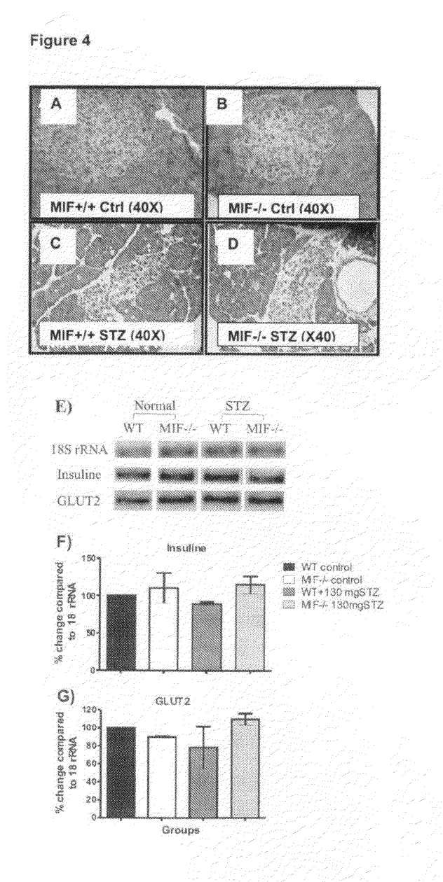

Progression of NIDDM is associated with involution of pancreatic islets and failure of β cells to secrete adequate amounts of insulin required to maintain normal blood glucose levels. GLUT2 is critical for glucose sensing by β cells in pancreas and therefore plays a critical role in regulating insulin secretion. Furthermore, GLUT2 mediates glucose-induced production of MIF by islet cells, which potentiates secretion of insulin (16). The present inventors therefore examined histopathological changes in pancreatic islets from diabetic WT and MIF−/− mice by microscopy (FIG. 4A). In addition, the present inventors quantified insulin and GLUT2 mRNA in pancreatic islet cells isolated from these mice by quantitative PCR (FIGS. 4B and 4C).

At week 8 after induction of NIDDM, both WT and MIF−/− mice showed involution of the pancreatic islets as compared to non-diabetic controls (FIG. 4A). However, at this time, no significant differences were noted in insulin and GLUT2 mRNA levels in pancreatic islets of WT and MIF−/− mice. MIF−/− mice displayed lower serum insulin levels than WT mice during the course of disease but these differences were not statistically significant. Together, these findings show that MIF deficiency does not lead to an increase in insulin production in pancreatic islet cells.

MIF−/− Mice Produce Significantly Less Resistin than WT Mice Following Induction of Diabetes

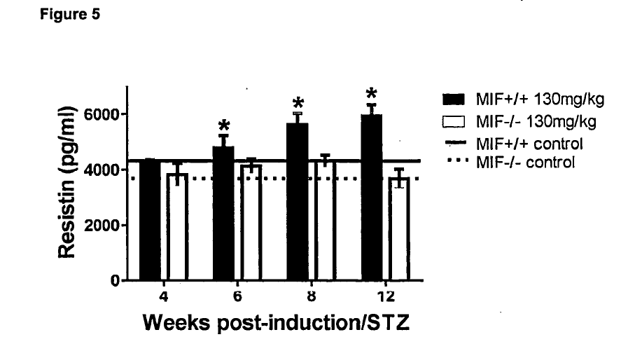

Resistin, which is produced by adipocytes, contributes to insulin resistance in NIDDM (17-24). The present inventors therefore compared serum resistin levels in WT and MIF−/− mice temporally following induction of NIDDM. MIF−/− mice consistently displayed significantly lower serum levels of resistin as compared to WT mice (FIG. 5). These findings indicate that MIF contributes to development of NIDDM at least in part by inducing resistin production in adipocytes.

Oral Administration of MIF Antagonist CPSI-1306 Impairs Inflammatory Cytokine Production and Reduces Blood Glucose Levels in Diabetic Mice.

To determine whether MIF is a potential therapeutic target in treatment of NIDDM, the present inventors administered MIF antagonist CSPI-1306 orally to ICR mice following STZ injection and examined its effect on the course of the disease. Mice treated with CSP156 showed a significant drop in their blood glucose levels which was associated with a reduction in serum levels of inflammatory cytokines. As expected, control mice treated with vehicle developed NIDDM characterized by high serum levels of glucose and inflammatory cytokines. These data demonstrate that MIF is a potential therapeutic target in management of NIDDM. Furthermore, they also show that orally bio-available MIF antagonists can be effective to treating this disease.

Levels of MIF, TNF-α and Resistin are Significantly Increased in Patients Suffering from NIDDM.

To examine the clinical relevance of the experimental findings, the present inventors measured serum levels of MIF, TNF-α and resistin in patients with NIDDM and compared them to normal healthy controls. Both males and females suffering from NIDDM displayed significantly higher serum levels of MIF, TNF-α and resistin as compared to their non-diabetic counterparts. These data indicate that MIF may contribute to pathogenesis of NIDDM in humans.

Discussion

Several experimental studies have implicated MIF in the pathogenesis of autoimmune insulin-dependent diabetes (IDDM) which also known as type I diabetes (10;11;25). The present inventors have found that MIF is involved in pathogenesis of NIDDM (type 2 DM) and is a therapeutic target in treatment of this disease.

Previous studies have shown that MIF, by virtue of its pro-inflammatory activity, plays a critical role in pathogenesis of inflammatory diseases such as arthritis (2; 26-29), atherosclerosis (2;30;31), asthma (32;33), glomerulonephritis (6) as well as IDDM which is associated with loss of endogenous insulin due to destruction of pancreatic R cells. Experimental studies inducing IDDM in MIF−/− mice by multiple low doses of STZ or using anti-MIF neutralizing antibody in NOD mice have shown that MIF deficiency attenuates development of IDDM (10;11). Conversely, Bojunga et al have found that administration of recombinant MIF to NOD mice increases incidence of diabetes (25). Although these findings suggest that MIF is involved in pathogenesis of IDDM, it is not clear whether MIF plays a similar role in development of NIDDM which is associated with insulin resistance and subsequent failure of pancreatic β cells to secrete adequate insulin.

MIF is produced by the cells of pancreatic islets and has been detected within granules containing insulin (34) and has been shown to enhanced glucose induced secretion of insulin in pancreas in an autocrine manner (34;35). Therefore, it has been hypothesized that MIF may play a beneficial role in diabetes by regulating glucose homeostasis by stimulating insulin secretion by β-cells (34;35) and by also modulating glucagon secretion (34;35). Nonetheless, previous studies have shown that serum levels of MIF are higher in patients with NIDDM (7;36-39). A study by Vozarova et al. has also reported a link between MIF levels and insulin resistance in individuals who are prone to NIDDM (36). Despite these findings it is not clear whether increased MIF production in NIDDM patients contributes to disease progression or is a secondary consequence of NIDDM. In the present study, the present inventors found that MIF deficiency resulted in a significant attenuation of STZ-induced NIDDM in mice. The present inventors also found that patients with NIDDM show a significant increase in serum MIF levels. Collectively, these findings indicate that MIF plays a detrimental role in NIDDM and contributes to pathogenesis of this disease. Interestingly, the present inventors also found that pancreatic islet cells from STZ-injected WT and MIF−/− mice contained comparable levels insulin and GLUT2 mRNA indicating that MIF deficiency has no effect on insulin production or glucose transporter 2 levels in this model.

Pro-inflammatory cytokines IL-1β and TNF-α have been shown to be involved in pathogenesis of NIDDM (18;40-42). Levels of these cytokines are elevated in obese individuals with high body mass index (BMI) who are more prone to develop NIDDM (18;40-42). These cytokines induce acute-phase reaction and cause adipose tissue inflammation resulting in an increase in secretion of inflammatory cytokines by adipocytes as well as release of adipokines such as resistin which is responsible for development of insulin resistance. In addition, IL-6 and TNF-α interfere with insulin signaling pathway and reduce responsiveness of muscle and hepatocytes to insulin (43). Previous studies have reported that high serum levels of MIF correlate with obesity and high BMI whereas increased physical activity and weight reduction is associated with a substantial reduction in MIF. Furthermore, polymorphism in MIF gene promoter has been linked to obesity (44). Taken together, these findings suggest that MIF can contribute to pathogenesis of NIDDM by inducing production of proinflammatory cytokines and/or by modulating adipocyte function and regulating production of adipokines such as resistin. In the current study, the present inventors observed that MIF−/− mice produced significantly less IL-1β, IL-6 and TNF-α as compared to their WT counterparts upon STZ injection. Furthermore, serum levels of resistin were significantly lower in MIF−/− mice. Together, the observations indicate that MIF contributes to pathogenesis of NIDDM by inducing production of inflammatory cytokines as well as resistin. This is perhaps not surprising since adipocytes produce MIF and it is likely that MIF released from the inflamed adipose tissue promotes development of NIDDM by inducing secretion of inflammatory cytokines as well as resistin from the adipocyte in an autocrine manner.

A recent study using MIF blocking antibodies has found that therapeutic blockade of MIF reduces severity and progression of autoimmune diabetes mellitus (11). Therefore, using an orally bioavailable MIF antagonist CPSI-1306, the present inventors determined whether MIF is a therapeutic target to treat STZ-induced NIDDM in outbred ICR mice. In deed, administration of CPSI-1306 (0.1 and 0.01 mg/kg) to ICR mice with STZ injection reduced severity of NIDDM which was associated with a significant reduction in serum levels of inflammatory cytokines and blood glucose and less polyuria. The stoppage of CPSI-1306 treatment resulted in a spike in blood glucose levels (data not shown). Taken together, these results demonstrate that MIF is a novel therapeutic target to treat NIDDM.

In conclusion, the present inventors have shown that deficiency of MIF significantly reduces severity and progression of STZ-induced NIDDM. Lack of MIF reduces production of pro-inflammatory cytokines as well as resistin but has no effect on insulin and GLUT2 expression in pancreas. Further, the present inventors show that blockade of MIF activity using a MIF antagonist reduces production of inflammatory cytokines and attenuates NIDDM in outbred ICR mice. Herder et al had reported that increased blood levels of MIF are associated with risk of developing NIDDM in females but not males. However, here the present inventors found that both male and female patients with NIDDM show a significant increase in MIF, TNF-α and resistin in their sera suggesting that gender may no influence the pathogenic role of MIF in NIDDM.

FIG. 1: MIF−/− BALB/c Mice Develop Significantly Less Severe NIDDM as Compared to WT BALB/c Mice Following STZ Injection.

Seven to eight weeks old sex matched WT and MIF−/− mice were injected with a single dose of STZ (130 mg/Kg) intraperitoneally to induce NIDDM. Progression of NIDDM was monitored by measuring blood glucose (A), weight loss (B), urine output (C) and food consumption (D) once a week, as described in the Materials and Methods. Control mice received intraperitoneal injection of PBS. The data are from one representative experiment out of three and is the mean of five to six animals per group at each time point. * p<0.05.

FIG. 2: MIF−/− BALB/c Mice Display Better Glucose Tolerance than WT Mice.

Four weeks following STZ injection, glucose tolerance of WT and MIF−/− BALB/c mice was determined as described before. The data are mean blood glucose levels (n=6 each group)+SE from a single experiment. Similar results were observed in two independent experiments. * p<0.05.

FIG. 3: Analysis of IL-1, IL-6 and TNF-α Production in WT and MIF−/− BALB/c Mice Following STZ-Induced NIDDM.

Following induction of NIDDM, WT and MIF−/− mice were bled once a week by tail snipping and levels of IL-1, IL-6 and TNF-α in sera were measured by ELISA. The data are mean serum levels in pg/ml (n=5-6 each group/per time point)+SE from one out of three experiments with similar results.

FIG. 4: Histopathology and Quantification of Insulin and GLUT2 mRNA Levels in Pancreas of WT and MIF−/− Mice.

At week 8 after injection of STZ, WT and MIF−/− mice were euthanized and histopathological changes in pancreatic islets were examined (A-D). At this time, pancreatic islets were isolated by collagenase digestion and discontinuous Ficoll-density gradient and mRNA levels of insulin (e and F) and GLUT2 (E and G) were measured by semi-quantitative PCR. The data is from one out of two experiments with similar results.

FIG. 5: MIF−/− Mice Produce Significantly Less Resistin as Compared to WT Mice.

Serum levels of resistin were measured by ELISA in WT and MIF−/− mice at weeks 4, 6, 8 and 12 following STZ injection. The data are mean serum levels in pg/ml (n=5-6 each group/per time point)+SE from one out of three experiments with similar results.

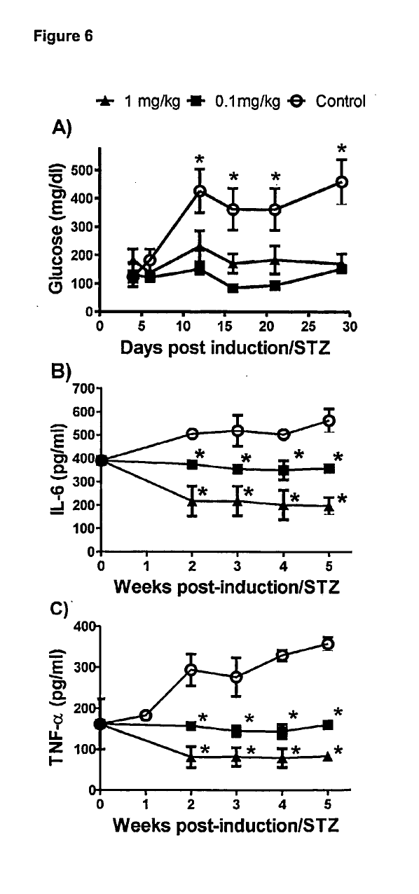

FIG. 6: Oral Administration of MIF Antagonist CPSI-1306 Significantly Reduces Severity and Progression of STZ-Induced NIDDM in Outbred ICR Mice.

NIDDM was induced in ICR mice by a single intraperitoneal injection of STZ (90 mg/kg). On day 5 post-STZ injection and thereafter mice were orally administered CPSI-1306 (1 mg/kg or 0.1 mg/Kg) daily for 30 days. Mice were bled once a week by tail sniping and levels of glucose (A) and inflammatory cytokines IL-6 (B) and TNF-α (C) in the blood were determined. The data are from one representative experiment out of three. * p<0.05.

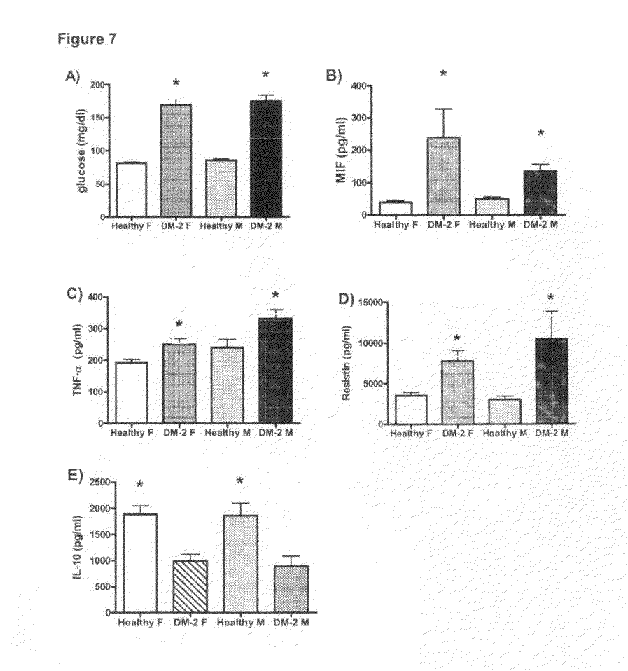

FIG. 7: Analysis of Blood Glucose, MIF, TNF-α and Resistin Levels in Patients with NIDDM.

Blood glucose levels (A) in patients (males n=27 and females n=46; age 35-65 years) diagnosed with NIDDM and healthy controls (males n=23 and females n-59; age 35-65 years) were measured by biochemical analysis. Serum levels of MIF (B), TNF-α (C) and resistin (D) were measured by ELISA. That data shown as mean levels +SE. * p<0.05.

The contents of each of the following references are hereby incorporated by reference.
1. Baugh, J. A., Chitnis, S., Donnelly, S. C., Monteiro, J., Lin, X., Plant, B. J., Wolfe, F., Gregersen, P. K., Bucala, R. (2002) A functional promoter polymorphism in the macrophage migration inhibitory factor (MIF) gene associated with disease severity in rheumatoid arthritis. Genes Immun. 3, 170-176.
2. Morand, E. F., Leech, M., Bernhagen, J. (2006) MIF: a new cytokine link between rheumatoid arthritis and atherosclerosis. Nat. Rev. Drug Discov. 5, 399-410.
3. Santos, L. L., Morand, E. F. (2009) Macrophage migration inhibitory factor: a key cytokine in RA, SLE and atherosclerosis. Clin. Chim. Acta 399, 1-74. de Jong, Y. P., Abadia-Molina, A. C., Satoskar, A. R., Clarke, K., Rietdijk, S. T., Faubion, W. A., Mizoguchi, E., Metz, C. N., Alsahli, M., ten Hove, T., Keates, A. C., Lubetsky, J. B., Farrell, R. J., Michetti, P., van Deventer, S. J., Lolis, E., David, J. R., Bhan, A. K., Terhorst, C. (2001) Development of chronic colitis is dependent on the cytokine MIF. Nat. Immunol. 2, 1061-1066.
5. Mikulowska, A., Metz, C. N., Bucala, R., Holmdahl, R. (1997) Macrophage migration inhibitory factor is involved in the pathogenesis of collagen type II-induced arthritis in mice. J. Immunol. 158, 5514-5517.

6. Hoi, A. Y., Hickey, M. J., Hall, P., Yamana, J., O'Sullivan, K. M., Santos, L. L., James, W. G., Kitching, A. R., Morand, E. F. (2006) Macrophage migration inhibitory factor deficiency attenuates macrophage recruitment, glomerulonephritis, and lethality in MRL/Ipr mice. J. Immunol. 177, 5687-5696.
7. Herder, C., Kolb, H., Koenig, W., Haastert, B., Muller-Scholze, S., Rathmann, W., Holle, R., Thorand, B., Wichmann, H. E. (2006) Association of systemic concentrations of macrophage migration inhibitory factor with impaired glucose tolerance and type 2 diabetes: results from the Cooperative Health Research in the Region of Augsburg, Survey 4 (KORA S4). Diabetes Care 29, 368-371.
8. Toso, C., Emamaullee, J. A., Merani, S., Shapiro, A. M. (2008) The role of macrophage migration inhibitory factor on glucose metabolism and diabetes. Diabetologia 51, 1937-1946.
9. Hanifi-Moghaddam, P., Schloot, N. C., Kappler, S., Seissler, J., Kolb, H. (2003) An association of autoantibody status and serum cytokine levels in type 1 diabetes. Diabetes 52, 1137-1142.
10. Cvetkovic, I., Al Abed, Y., Miljkovic, D., Maksimovic-Ivanic, D., Roth, J., Bacher, M., Lan, H. Y., Nicoletti, F., Stosic-Grujicic, S. (2005) Critical role of macrophage migration inhibitory factor activity in experimental autoimmune diabetes. Endocrinology 146, 2942-2951.
11. Stosic-Grujicic, S., Stojanovic, I., Maksimovic-Ivanic, D., Momcilovic, M., Popadic, D., Harhaji, L., Miljkovic, D., Metz, C., Mangano, K., Papaccio, G., Al Abed, Y., Nicoletti, F. (2008) Macrophage migration inhibitory factor (MIF) is necessary for progression of autoimmune diabetes mellitus. J. Cell Physiol 215, 665-675.
12. Bozza, M., Satoskar, A. R., Lin, G., Lu, B., Humbles, A. A., Gerard, C., David, J. R. (1999) Targeted disruption of migration inhibitory factor gene reveals its critical role in sepsis. J. Exp. Med. 189, 341-346.
13. Hayashi, K., Kojima, R., Ito, M. (2006) Strain differences in the diabetogenic activity of streptozotocin in mice. Biol. Pharm. Bull. 29, 1110-1119.
14. Ito, M., Kondo, Y., Nakatani, A., Naruse, A. (1999) New model of progressive non-insulin-dependent diabetes mellitus in mice induced by streptozotocin. Biol. Pharm. Bull. 22, 988-989.
15. Ito, M., Kondo, Y., Nakatani, A., Hayashi, K., Naruse, A. (2001) Characterization of low dose streptozotocin-induced progressive diabetes in mice. Environ. Toxicol Pharmacol. 9, 71-78.
16. Sakaue, S., Nishihira, J., Hirokawa, J., Yoshimura, H., Honda, T., Aoki, K., Tagami, S., Kawakami, Y. (1999) Regulation of macrophage migration inhibitory factor (MIF) expression by glucose and insulin in adipocytes in vitro. Mol. Med. 5, 361-371.
17. Adeghate, E. (2004) An update on the biology and physiology of resistin. Cell Mol. Life Sci. 61, 2485-2496.
18. Alexandraki, K., Piperi, C., Kalofoutis, C., Singh, J., Alaveras, A., Kalofoutis, A. (2006) Inflammatory process in type 2 diabetes: The role of cytokines. Ann. N.Y. Acad. Sci. 1084, 89-117.
19. Arner, P. (2005) Insulin resistance in type 2 diabetes—role of the adipokines. Curr. Mol. Med. 5, 333-339.
20. Barnes, K. M., Miner, J. L. (2009) Role of resistin in insulin sensitivity in rodents and humans. Curr. Protein Pept. Sci. 10, 96-107.
21. Goldstein, B. J. (2002) Insulin resistance as the core defect in type 2 diabetes mellitus. Am. J. Cardiol. 90, 3G-10G.
22. Kochan, Z., Karbowska, J. (2003) [Resistine—a new hormone secreted by adipose tissue (adipose tissue in insulin resistance)]. Przegl. Lek. 60, 40-42.
23. McTernan, P. G., Fisher, F. M., Valsamakis, G., Chetty, R., Harte, A., McTernan, C. L., Clark, P. M., Smith, S. A., Barnett, A. H., Kumar, S. (2003) Resistin and type 2 diabetes: regulation of resistin expression by insulin and rosiglitazone and the effects of recombinant resistin on lipid and glucose metabolism in human differentiated adipocytes. J. Clin. Endocrinol. Metab 88, 6098-6106.
24. Mojiminiyi, 0. A., Abdella, N. A. (2007) Associations of resistin with inflammation and insulin resistance in patients with type 2 diabetes mellitus. Scand. J. Clin. Lab Invest 67, 215-225.
25. Bojunga, J., Kusterer, K., Bacher, M., Kurek, R., Usadel, K. H., Renneberg, H. (2003) Macrophage migration inhibitory factor and development of type-1 diabetes in non-obese diabetic mice. Cytokine 21, 179-186.
26. Ayoub, S., Hickey, M. J., Morand, E. F. (2008) Mechanisms of disease: macrophage migration inhibitory factor in SLE, RA and atherosclerosis. Nat. Clin. Pract. Rheumatol. 4, 98-105.
27. de Jager, W., Hoppenreijs, E. P., Wulffraat, N. M., Wedderburn, L. R., Kuis, W., Prakken, B. J. (2007) Blood and synovial fluid cytokine signatures in patients with juvenile idiopathic arthritis: a cross-sectional study. Ann. Rheum. Dis. 66, 589-598.
28. Leech, M., Metz, C., Hall, P., Hutchinson, P., Gianis, K., Smith, M., Weedon, H., Holdsworth, S. R., Bucala, R., Morand, E. F. (1999) Macrophage migration inhibitory factor in rheumatoid arthritis: evidence of proinflammatory function and regulation by glucocorticoids. Arthritis Rheum. 42, 1601-1608.
29. Morand, E. F., Leech, M., Weedon, H., Metz, C., Bucala, R., Smith, M. D. (2002) Macrophage migration inhibitory factor in rheumatoid arthritis: clinical correlations. Rheumatology. (Oxford) 41, 558-562.
30. Burger-Kentischer, A., Goebel, H., Seiler, R., Fraedrich, G., Schaefer, H. E., Dimmeler, S., Kleemann, R., Bernhagen, J., Ihling, C. (2002) Expression of macrophage migration inhibitory factor in different stages of human atherosclerosis. Circulation 105, 1561-1566.
31. Chen, Z., Sakuma, M., Zago, A. C., Zhang, X., Shi, C., Leng, L., Mizue, Y., Bucala, R., Simon, D. (2004) Evidence for a role of macrophage migration inhibitory factor in vascular disease. Arterioscler. Thromb. Vasc. Biol. 24, 709-714.
32. Mizue, Y., Ghani, S., Leng, L., McDonald, C., Kong, P., Baugh, J., Lane, S. J., Craft, J., Nishihira, J., Donnelly, S. C., Zhu, Z., Bucala, R. (2005) Role for macrophage migration inhibitory factor in asthma. Proc. Natl. Acad. Sci. U.S.A 102, 14410-14415.
33. Wang, B., Huang, X., Wolters, P. J., Sun, J., Kitamoto, S., Yang, M., Riese, R., Leng, L., Chapman, H. A., Finn, P. W., David, J. R., Bucala, R., Shi, G. P. (2006) Cutting edge: Deficiency of macrophage migration inhibitory factor impairs murine airway allergic responses. J. Immunol. 177, 5779-5784.
34. Waeber, G., Calandra, T., Roduit, R., Haefliger, J. A., Bonny, C., Thompson, N., Thorens, B., Temler, E., Meinhardt, A., Bacher, M., Metz, C. N., Nicod, P., Bucala, R. (1997) Insulin secretion is regulated by the glucose-dependent production of islet beta cell macrophage migration inhibitory factor. Proc. Natl. Acad. Sci. U.S.A 94, 4782-4787.
35. Waeber, G., Calandra, T., Bonny, C., Bucala, R. (1999) A role for the endocrine and pro-inflammatory mediator MIF in the control of insulin secretion during stress. Diabetes Metab Res Rev. 15, 47-54.
36. Vozarova, B., Stefan, N., Hanson, R., Lindsay, R. S., Bogardus, C., Tataranni, P. A., Metz, C., Bucala, R. (2002) Plasma concentrations of macrophage migration inhibitory factor are elevated in Pima Indians compared to Caucasians and are associated with insulin resistance. Diabetologia 45, 1739-1741.
37. Herder, C., Klopp, N., Baumert, J., Muller, M., Khuseyinova, N., Meisinger, C., Martin, S., Illig, T., Koenig, W., Thorand, B. (2008) Effect of macrophage migration inhibitory factor (MIF) gene variants and MIF serum concentrations on the risk of type 2 diabetes: results from the MONICA/KORA Augsburg Case-Cohort Study, 1984-2002. Diabetologia 51, 276-284.
38. Herder, C., Illig, T., Baumert, J., Muller, M., Klopp, N., Khuseyinova, N., Meisinger, C., Martin, S., Thorand, B., Koenig, W. (2008) Macrophage migration inhibitory factor (MIF) and risk for coronary heart disease: results from the MONICA/KORA Augsburg case-cohort study, 1984-2002. Atherosclerosis 200, 380-388.
39. Herder, C., Peltonen, M., Koenig, W., Kraft, I., Muller-Scholze, S., Martin, S., Lakka, T., Ilanne-Parikka, P., Eriksson, J. G., Hamalainen, H., Keinanen-Kiukaanniemi, S., Valle, T. T., Uusitupa, M., Lindstrom, J., Kolb, H., Tuomilehto, J. (2006) Systemic immune mediators and lifestyle changes in the prevention of type 2 diabetes: results from the Finnish Diabetes Prevention Study. Diabetes 55, 2340-2346.
40. Atsumi, T., Cho, Y. R., Leng, L., McDonald, C., Yu, T., Danton, C., Hong, E. G., Mitchell, R. A., Metz, C., Niwa, H., Takeuchi, J., Onodera, S., Umino, T., Yoshioka, N., Koike, T., Kim, J. K., Bucala, R. (2007) The proinflammatory cytokine macrophage migration inhibitory factor regulates glucose metabolism during systemic inflammation. J. Immunol. 179, 5399-5406.
41. Bastard, J. P., Maachi, M., Lagathu, C., Kim, M. J., Caron, M., Vidal, H., Capeau, J., Feve, B. (2006) Recent advances in the relationship between obesity, inflammation, and insulin resistance. Eur. Cytokine Netw. 17, 4-12.
42. Crook, M. (2004) Type 2 diabetes mellitus: a disease of the innate immune system? An update. Diabet. Med. 21, 203-207.
43. Fain, J. N. (2006) Release of interleukins and other inflammatory cytokines by human adipose tissue is enhanced in obesity and primarily due to the nonfat cells. Vitam. Horm. 74, 443-477.
44. Sakaue, S., Ishimaru, S., Hizawa, N., Ohtsuka, Y., Tsujino, I., Honda, T., Suzuki, J., Kawakami, Y., Nishihira, J., Nishimura, M. (2006) Promoter polymorphism in the macrophage migration inhibitory factor gene is associated with obesity. Int. J. Obes. (Lond) 30, 238-242.

The entire contents of U.S. application Ser. Nos. 10/927,494, filed Aug. 27, 2004; 11/090,128, filed Mar. 28, 2005; and U.S. Provisional Application No. 61/264,620, filed Nov. 25, 2009; and PCT Application No. PCT/US10/58135, filed Nov. 26, 2010, are independently incorporated herein by reference.

Non-limiting examples of subject compounds that may be used herein, and methods of making same, may be found in U.S. application Ser. No. 11/090,128, filed Mar. 28, 2005; U.S. Provisional Application No. 61/264,406, filed Nov. 25, 2009; and PCT Application No. PCT/US10/58135, filed Nov. 26, 2010.

This application is based on and claims priority to U.S. Provisional Application 61/264,620, filed Nov. 25, 2009, the entire contents of which are hereby incorporated by reference.

Other embodiments, which are not intended to be limiting, are described below.

1. A method for treating diabetes in a subject, comprising administering to said subject a compound having the following formula: 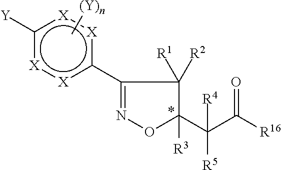
wherein at least the carbon marked “*” is chiral;
wherein R¹, R², R³, R⁴, and R⁵ are each independently hydrogen, an alkyl group, a cycloalkyl group, a halo group, an alkenyl group, an alkynyl group, a hydroxy group, an oxo group, a mercapto group, an alkylthio group, an alkoxy group, an aryl group, a heterocyclic group, a heteroaryl group, an aryloxy group, a heteroaryloxy group, an aralkyl group, a heteroaralkyl group, an aralkoxy group, a heteroaralkoxy group, an amino group, an alkylamino group, a dialkylamino group, an amidine group, an amide group, a carbamoyl group, an alkylcarbonyl group, an alkoxycarbonyl group, an alkylaminocarbonyl group, a dialkylamino carbonyl group, an arylcarbonyl group, an aryloxycarbonyl group, an alkylsulfonyl group, an arylsulfonyl group, perhaloalkyl group, a perhaloalkoxy group, a perhalocycloalkyl group, a perhaloalkenyl group, a perhaloalkynyl group, a perhaloaryl group, or a perhaloaralkyl group; wherein R¹ and R² may be taken together to form a cyclic group; wherein R⁴ and R⁵ may be taken together to form a cyclic group; wherein each group may be optionally and independently straight or branched; wherein each group may be optionally and independently substituted by one or more independent substituents; and wherein one or more than one atom in each group may be optionally and independently replaced with one or more independent heteroatoms;
wherein each X is independently carbon or nitrogen, wherein when any X is carbon, it comprises a Y substituent, n being an integer of from 1 to 4 and being the number of X's that are carbon;
wherein each Y is independently a carbonyl group, a carboxylic acid group, a carboxylate group, hydrogen, an alkyl group, a cycloalkyl group, a halo group, an alkenyl group, an alkynyl group, a hydroxy group, an oxo group, a mercapto group, an alkylthio group, an alkoxy group, an aryl group, a heterocyclic group, a heteroaryl group, an aryloxy group, a heteroaryloxy group, an aralkyl group, a heteroaralkyl group, an aralkoxy group, a heteroaralkoxy group, an amino group, an alkylamino group, a dialkylamino group, an amidine group, an amide group, a carbamoyl group, an alkylcarbonyl group, an alkoxycarbonyl group, an alkylaminocarbonyl group, a dialkylamino carbonyl group, an arylcarbonyl group, an aryloxycarbonyl group, an alkylsulfonyl group, an arylsulfonyl group, perhaloalkyl group, a perhaloalkoxy group, a perhalocycloalkyl group, a perhaloalkenyl group, a perhaloalkynyl group, a perhaloaryl group, or a perhaloaralkyl group; wherein two Y groups may be taken together to form a cyclic or aryl group; wherein each group may be optionally and independently straight or branched; wherein each group may be optionally and independently substituted by one or more independent substituents; and wherein one or more than one atom in each group may be optionally and independently replaced with one or more independent heteroatoms;
and wherein R¹⁶ is an alkyl group, a cycloalkyl group, a halo group, an alkenyl group, an alkynyl group, a hydroxy group, an oxo group, a mercapto group, an alkylthio group, an alkoxy group, an aryl group, a heterocyclic group, a heteroaryl group, an aryloxy group, a heteroaryloxy group, an aralkyl group, a heteroaralkyl group, an aralkoxy group, a heteroaralkoxy group, an amino group, an alkylamino group, a dialkylamino group, an amidine group, an amide group, a carbamoyl group, an alkylcarbonyl group, an alkoxycarbonyl group, an alkylaminocarbonyl group, a dialkylamino carbonyl group, an arylcarbonyl group, an aryloxycarbonyl group, an alkylsulfonyl group, an arylsulfonyl group, perhaloalkyl group, a perhaloalkoxy group, a perhalocycloalkyl group, a perhaloalkenyl group, a perhaloalkynyl group, a perhaloaryl group, or a perhaloaralkyl group; wherein any two alkyl groups may be taken together to form a cyclic group; wherein each group may be optionally and independently straight or branched; wherein each group may be optionally and independently substituted by one or more independent substituents; and wherein one or more than one atom in each group may be optionally and independently replaced with one or more independent heteroatoms;
or salt thereof, prodrug thereof, or combination thereof, optionally in contact with one or more pharmaceutical carrier.

2. A method for reducing the blood glucose level of a subject suffering from diabetes, comprising administering to said subject a compound having the following formula: 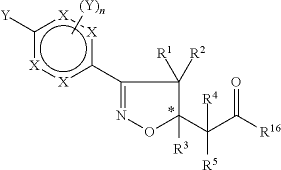
wherein at least the carbon marked “*” is chiral;
wherein R¹, R², R³, R⁴, and R⁵ are each independently hydrogen, an alkyl group, a cycloalkyl group, a halo group, an alkenyl group, an alkynyl group, a hydroxy group, an oxo group, a mercapto group, an alkylthio group, an alkoxy group, an aryl group, a heterocyclic group, a heteroaryl group, an aryloxy group, a heteroaryloxy group, an aralkyl group, a heteroaralkyl group, an aralkoxy group, a heteroaralkoxy group, an amino group, an alkylamino group, a dialkylamino group, an amidine group, an amide group, a carbamoyl group, an alkylcarbonyl group, an alkoxycarbonyl group, an alkylaminocarbonyl group, a dialkylamino carbonyl group, an arylcarbonyl group, an aryloxycarbonyl group, an alkylsulfonyl group, an arylsulfonyl group, perhaloalkyl group, a perhaloalkoxy group, a perhalocycloalkyl group, a perhaloalkenyl group, a perhaloalkynyl group, a perhaloaryl group, or a perhaloaralkyl group; wherein R¹ and R² may be taken together to form a cyclic group; wherein R⁴ and R⁵ may be taken together to form a cyclic group; wherein each group may be optionally and independently straight or branched; wherein each group may be optionally and independently substituted by one or more independent substituents; and wherein one or more than one atom in each group may be optionally and independently replaced with one or more independent heteroatoms;
wherein each X is independently carbon or nitrogen, wherein when any X is carbon, it comprises a Y substituent, n being an integer of from 1 to 4 and being the number of X's that are carbon;
wherein each Y is independently a carbonyl group, a carboxylic acid group, a carboxylate group, hydrogen, an alkyl group, a cycloalkyl group, a halo group, an alkenyl group, an alkynyl group, a hydroxy group, an oxo group, a mercapto group, an alkylthio group, an alkoxy group, an aryl group, a heterocyclic group, a heteroaryl group, an aryloxy group, a heteroaryloxy group, an aralkyl group, a heteroaralkyl group, an aralkoxy group, a heteroaralkoxy group, an amino group, an alkylamino group, a dialkylamino group, an amidine group, an amide group, a carbamoyl group, an alkylcarbonyl group, an alkoxycarbonyl group, an alkylaminocarbonyl group, a dialkylamino carbonyl group, an arylcarbonyl group, an aryloxycarbonyl group, an alkylsulfonyl group, an arylsulfonyl group, perhaloalkyl group, a perhaloalkoxy group, a perhalocycloalkyl group, a perhaloalkenyl group, a perhaloalkynyl group, a perhaloaryl group, or a perhaloaralkyl group; wherein two Y groups may be taken together to form a cyclic or aryl group; wherein each group may be optionally and independently straight or branched; wherein each group may be optionally and independently substituted by one or more independent substituents; and wherein one or more than one atom in each group may be optionally and independently replaced with one or more independent heteroatoms;
and wherein R¹⁶ is an alkyl group, a cycloalkyl group, a halo group, an alkenyl group, an alkynyl group, a hydroxy group, an oxo group, a mercapto group, an alkylthio group, an alkoxy group, an aryl group, a heterocyclic group, a heteroaryl group, an aryloxy group, a heteroaryloxy group, an aralkyl group, a heteroaralkyl group, an aralkoxy group, a heteroaralkoxy group, an amino group, an alkylamino group, a dialkylamino group, an amidine group, an amide group, a carbamoyl group, an alkylcarbonyl group, an alkoxycarbonyl group, an alkylaminocarbonyl group, a dialkylamino carbonyl group, an arylcarbonyl group, an aryloxycarbonyl group, an alkylsulfonyl group, an arylsulfonyl group, perhaloalkyl group, a perhaloalkoxy group, a perhalocycloalkyl group, a perhaloalkenyl group, a perhaloalkynyl group, a perhaloaryl group, or a perhaloaralkyl group; wherein any two alkyl groups may be taken together to form a cyclic group; wherein each group may be optionally and independently straight or branched; wherein each group may be optionally and independently substituted by one or more independent substituents; and wherein one or more than one atom in each group may be optionally and independently replaced with one or more independent heteroatoms;
or salt thereof, prodrug thereof, or combination thereof, optionally in contact with one or more pharmaceutical carrier.

3. The method according to claim 1, wherein the subject suffers from type 2 diabetes.

4. A method for reducing or preventing an increase in the level of resistin in a subject, comprising administering to said subject a compound having the following formula: 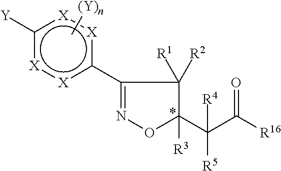
wherein at least the carbon marked “*” is chiral;
wherein R¹, R², R³, R⁴, and R⁵ are each independently hydrogen, an alkyl group, a cycloalkyl group, a halo group, an alkenyl group, an alkynyl group, a hydroxy group, an oxo group, a mercapto group, an alkylthio group, an alkoxy group, an aryl group, a heterocyclic group, a heteroaryl group, an aryloxy group, a heteroaryloxy group, an aralkyl group, a heteroaralkyl group, an aralkoxy group, a heteroaralkoxy group, an amino group, an alkylamino group, a dialkylamino group, an amidine group, an amide group, a carbamoyl group, an alkylcarbonyl group, an alkoxycarbonyl group, an alkylaminocarbonyl group, a dialkylamino carbonyl group, an arylcarbonyl group, an aryloxycarbonyl group, an alkylsulfonyl group, an arylsulfonyl group, perhaloalkyl group, a perhaloalkoxy group, a perhalocycloalkyl group, a perhaloalkenyl group, a perhaloalkynyl group, a perhaloaryl group, or a perhaloaralkyl group; wherein R¹ and R² may be taken together to form a cyclic group; wherein R⁴ and R⁵ may be taken together to form a cyclic group; wherein each group may be optionally and independently straight or branched; wherein each group may be optionally and independently substituted by one or more independent substituents; and wherein one or more than one atom in each group may be optionally and independently replaced with one or more independent heteroatoms;
wherein each X is independently carbon or nitrogen, wherein when any X is carbon, it comprises a Y substituent, n being an integer of from 1 to 4 and being the number of X's that are carbon;
wherein each Y is independently a carbonyl group, a carboxylic acid group, a carboxylate group, hydrogen, an alkyl group, a cycloalkyl group, a halo group, an alkenyl group, an alkynyl group, a hydroxy group, an oxo group, a mercapto group, an alkylthio group, an alkoxy group, an aryl group, a heterocyclic group, a heteroaryl group, an aryloxy group, a heteroaryloxy group, an aralkyl group, a heteroaralkyl group, an aralkoxy group, a heteroaralkoxy group, an amino group, an alkylamino group, a dialkylamino group, an amidine group, an amide group, a carbamoyl group, an alkylcarbonyl group, an alkoxycarbonyl group, an alkylaminocarbonyl group, a dialkylamino carbonyl group, an arylcarbonyl group, an aryloxycarbonyl group, an alkylsulfonyl group, an arylsulfonyl group, perhaloalkyl group, a perhaloalkoxy group, a perhalocycloalkyl group, a perhaloalkenyl group, a perhaloalkynyl group, a perhaloaryl group, or a perhaloaralkyl group; wherein two Y groups may be taken together to form a cyclic or aryl group; wherein each group may be optionally and independently straight or branched; wherein each group may be optionally and independently substituted by one or more independent substituents; and wherein one or more than one atom in each group may be optionally and independently replaced with one or more independent heteroatoms;
and wherein R¹⁶ is an alkyl group, a cycloalkyl group, a halo group, an alkenyl group, an alkynyl group, a hydroxy group, an oxo group, a mercapto group, an alkylthio group, an alkoxy group, an aryl group, a heterocyclic group, a heteroaryl group, an aryloxy group, a heteroaryloxy group, an aralkyl group, a heteroaralkyl group, an aralkoxy group, a heteroaralkoxy group, an amino group, an alkylamino group, a dialkylamino group, an amidine group, an amide group, a carbamoyl group, an alkylcarbonyl group, an alkoxycarbonyl group, an alkylaminocarbonyl group, a dialkylamino carbonyl group, an arylcarbonyl group, an aryloxycarbonyl group, an alkylsulfonyl group, an arylsulfonyl group, perhaloalkyl group, a perhaloalkoxy group, a perhalocycloalkyl group, a perhaloalkenyl group, a perhaloalkynyl group, a perhaloaryl group, or a perhaloaralkyl group; wherein any two alkyl groups may be taken together to form a cyclic group; wherein each group may be optionally and independently straight or branched; wherein each group may be optionally and independently substituted by one or more independent substituents; and wherein one or more than one atom in each group may be optionally and independently replaced with one or more independent heteroatoms;
or salt thereof, prodrug thereof, or combination thereof, optionally in contact with one or more pharmaceutical carrier.

5. The method according to claim 4, wherein the subject suffers from diabetes.

6. The method according to claim 4, wherein the subject suffers from type 2 diabetes.

7. The method according to claim 1, wherein the compound having the following formula 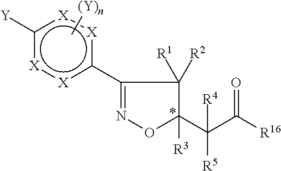
is selected from the following compounds:

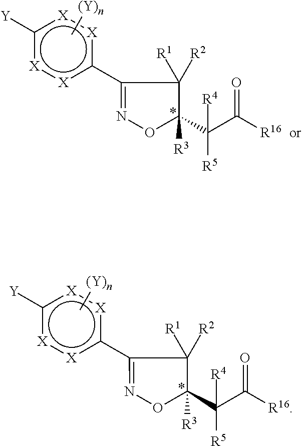

8. The method according to claim 1, wherein R¹, R², R³, R⁴, and R⁵ are each independently hydrogen, an alkyl group, a cycloalkyl group, an alkenyl group, an alkynyl group, an oxo group, an aryl group, a heterocyclic group, a heteroaryl group, an aralkyl group, a heteroaralkyl group, an amino group, an alkylamino group, a dialkylamino group, an amidine group, an amide group, an alkylcarbonyl group, an alkoxycarbonyl group, an alkylaminocarbonyl group, a dialkylamino carbonyl group, an arylcarbonyl group, an aryloxycarbonyl group, an alkylsulfonyl group, an arylsulfonyl group, perhaloalkyl group, a perhalocycloalkyl group, a perhaloalkenyl group, a perhaloalkynyl group, a perhaloaryl group, or a perhaloaralkyl group; wherein R¹ and R² may be taken together to form a cyclic group; wherein R⁴ and R⁵ may be taken together to form a cyclic group; wherein each group may be optionally and independently straight or branched; wherein each group may be optionally and independently substituted by one or more independent substituents; and wherein one or more than one atom in each group may be optionally and independently replaced with one or more independent heteroatoms.

9. The method according to claim 1, wherein one or both of R⁴ and R⁵ are hydrogen.

10. The method according to claim 1, wherein only one of R⁴ and R⁵ is hydrogen.

11. The method according to claim 1, wherein the structure 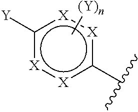
is selected from one of the following three structures: 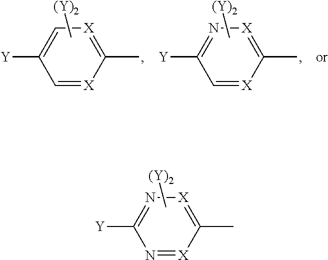
wherein each X is independently carbon or nitrogen, and wherein X is carbon, it independently comprises a Y substituent. In the three structures shown above, in one embodiment, the X's may be carbon, each carbon independently comprising a Y substituent.

12. The method according to claim 1, wherein 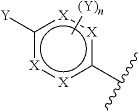
is selected from one of the following structures:

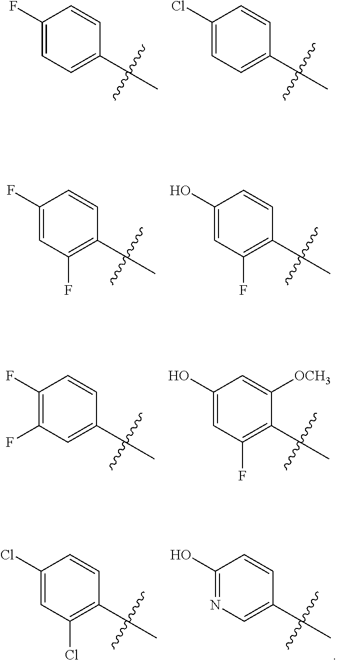

13. The method according to claim 1, wherein Y is an alkyl group, a cycloalkyl group, a halo group, a perfluoroalkyl group, a perfluoroalkoxy group, an alkenyl group, an alkynyl group, a hydroxy group, an oxo group, a mercapto group, an alkylthio group, an alkoxy group, an aryl group, a heteraryl group, an aryloxy group, a heteroaryloxy group, an aralkyl group, a heteroaralkyl group, an aralkoxy group, a heteroaralkoxy group, an HO—(C═O)— group, an amino group, an alkylamino group, a dialkylamino group, a carbamoyl group, an alkylcarbonyl group, an alkoxycarbonyl group, an alkylaminocarbonyl group, a dialkylamino carbonyl group, an arylcarbonyl group, an aryloxycarbonyl group, an alkylsulfonyl group, or an arylsulfonyl group, or have the following structure: 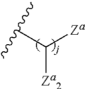
in which each Z^{a} is independently either hydrogen, hydroxyl, halogen, or a substituent group; and
“j” is independently either zero or an integer from one to four.

14. The method according to claim 1, wherein the structure: 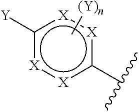
has the following structure: 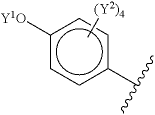
in which each Y¹ is independently a hydrogen or (C₁-C₆)alkyl; and
each Y² is independently a Y¹, hydroxyl group, halo group, —N₃, —CN, —SH, or —N(Y¹)₂.

15. The method according to claim 1, wherein the compound has the following structure: 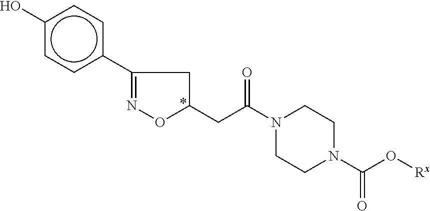
wherein R^{X} is a (C₁-C₆)alkyl, (C₂-C₆)alkenyl, (C₂-C₆)alkynyl, phenyl, (C₅-C₁₄)aryl, (C₄-C₁₄)heteroaryl, (C₂-C₁₄)heterocyclic or (C₃-C₁₀)cycloalkyl group.

16. The method according to claim 1, wherein the compound has the following structure: 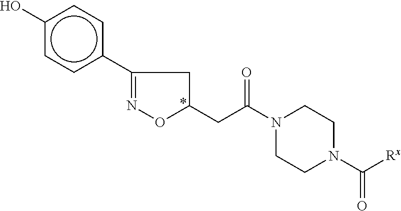
wherein R^{X} is a (C₁-C₆)alkyl, (C₂-C₆)alkenyl, (C₂-C₆)alkynyl, phenyl, (C₅-C₁₄)aryl, (C₄-C₁₄)heteroaryl, (C₂-C₁₄)heterocyclic or (C₃-C₁₀)cycloalkyl group.

17. The method according to claim 1, wherein the compound has the following structure: 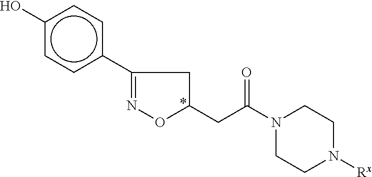
wherein R^{X} is a sulfonyl, carbonyl, (C₁-C₆)alkylsulfonyl, (C₁-C₆)alkylcarbonyl, (C₅-C₁₄)arylsulfonyl, (C₅-C₁₄)arylcarbonyl, (C₁-C₆)alkyl, (C₂-C₆)alkenyl, (C₂-C₆)alkynyl, phenyl, (C₅-C₁₄)aryl, (C₄-C₁₄)heteroaryl, (C₂-C₁₄)heterocyclic or (C₃-C₁₀)cycloalkyl group.

18. The method according to claim 1, wherein the compound has the following structure:

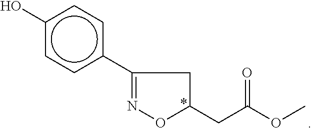

19. The method according to claim 1, wherein the compound has the following structure: 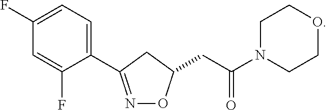 [SMILES: O=C(C[C@H]1CC(c2ccc(F)cc2F)=NO1)N1CCOCC1]
